(19) United States
(12) Patent Application Publication
(10) Pub. No.: US 2024/0407794 A1
(43) Pub. Date: Dec. 12, 2024

(54) DEVICES AND METHODS FOR ACCESSING A NEUROVASCULAR SITE

(71) Applicant: Imperative Care, Inc., Campbell, CA (US)

(72) Inventors: Miranda M. Ray, San Jose, CA (US); Kyle Alexander Godwin, Gardnerville, NV (US)

(21) Appl. No.: 18/737,390

(22) Filed: Jun. 7, 2024

Related U.S. Application Data
(60) Provisional application No. 63/472,244, filed on Jun. 9, 2023.

(51) Int. Cl. A61B 17/22; A61B 17/00; A61M 25/01

(52) U.S. Cl. CPC A61B 17/22; A61B 2017/00292; A61B 2017/22079; A61M 2025/0166

(57) ABSTRACT

A neurovascular catheter is disclosed for insertion into an internal carotid artery of a patient. The neurovascular catheter includes a flexible distal segment for insertion into an internal carotid artery of a patient. The flexible distal segment can include a distal portion, a transitional portion, and a support portion. The distal portion includes a tracking tip configured to be positioned within a cavernous segment of the internal carotid artery, and an increasing stiffness portion. The transitional portion can be positioned within a petrous segment of the internal carotid artery. The support portion can extend proximally to a base of a patient's skull.

### PRIORITY INFORMATION

This application claims the priority benefit of U.S. Provisional Application No. 63/472,244 filed Jun. 9, 2023, the entire contents of which are hereby incorporated by reference herein.

### BACKGROUND

Stroke is the third most common cause of death in the United States and the most disabling neurologic disorder. Approximately 700,000 patients suffer from stroke annually. Stroke is a syndrome characterized by the acute onset of a neurological deficit that persists for at least 24 hours, reflecting focal involvement of the central nervous system, and is the result of a disturbance of the cerebral circulation. Its incidence increases with age. Risk factors for stroke include systolic or diastolic hypertension, hypercholesterolemia, cigarette smoking, heavy alcohol consumption, and oral contraceptive use.

Hemorrhagic stroke accounts for 20% of the annual stroke population. Hemorrhagic stroke often occurs due to a rupture of an aneurysm or arteriovenous malformation bleeding into the brain tissue, resulting in cerebral infarction. The remaining 80% of the stroke population are ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. When a patient presents with neurological symptoms and signs which resolve completely within 1 hour, the term transient ischemic attack (TIA) is used. Etiologically, TIA and stroke share the same pathophysiologic mechanisms and thus represent a continuum based on persistence of symptoms and extent of ischemic insult.

Emboli occasionally form around the valves of the heart or in the left atrial appendage during periods of irregular heart rhythm and then are dislodged and follow the blood flow into the distal regions of the body. Those emboli can pass to the brain and cause an embolic stroke. As will be discussed below, many such occlusions occur in the middle cerebral artery (MCA), although such is not the only site where emboli come to rest.

When a patient presents with neurological deficit, a diagnostic hypothesis for the cause of stroke can be generated based on the patient's history, a review of stroke risk factors, and a neurologic examination. If an ischemic event is suspected, a clinician can tentatively assess whether the patient has a cardiogenic source of emboli, large artery extracranial or intracranial disease, small artery intraparenchymal disease, or a hematologic or other systemic disorder. A head CT scan is often performed to determine whether the patient has suffered an ischemic or hemorrhagic insult. Blood would be present on the CT scan in subarachnoid hemorrhage, intraparenchymal hematoma, or intraventricular hemorrhage.

Traditionally, emergent management of acute ischemic stroke consisted mainly of general supportive care, e.g. hydration, monitoring neurological status, blood pressure control, and/or anti-platelet or anti-coagulation therapy. In 1996, the Food and Drug Administration approved the use of Genentech Inc.'s thrombolytic drug, tissue plasminogen activator (t-PA) or Activase®, for treating acute stroke. A randomized, double-blind trial, the National Institute of Neurological Disorders and t-PA Stroke Study, revealed a statistically significant improvement in stoke scale scores at 24 hours in the group of patients receiving intravenous t-PA within 3 hours of the onset of an ischemic stroke. Since the approval of t-PA, an emergency room physician could, for the first time, offer a stroke patient an effective treatment besides supportive care.

However, treatment with systemic t-PA is associated with increased risk of intracerebral hemorrhage and other hemorrhagic complications. Patients treated with t-PA were more likely to sustain a symptomatic intracerebral hemorrhage during the first 36 hours of treatment. The frequency of symptomatic hemorrhage increases when t-PA is administered beyond 3 hours from the onset of a stroke. Besides the time constraint in using t-PA in acute ischemic stroke, other contraindications include the following: if the patient has had a previous stroke or serious head trauma in the preceding 3 months, if the patient has a systolic blood pressure above 185 mm Hg or diastolic blood pressure above 110 mmHg, if the patient requires aggressive treatment to reduce the blood pressure to the specified limits, if the patient is taking anticoagulants or has a propensity to hemorrhage, and/or if the patient has had a recent invasive surgical procedure. Therefore, only a small percentage of selected stroke patients are qualified to receive t-PA.

Obstructive emboli have also been mechanically removed from various sites in the vasculature for years. Mechanical therapies have involved capturing and removing the clot, dissolving the clot, disrupting and suctioning the clot, and/or creating a flow channel through the clot. One of the first mechanical devices developed for stroke treatment is the MERCI Retriever System (Concentric Medical, Redwood City, Calif.). A balloon-tipped guide catheter is used to access the internal carotid artery (ICA) from the femoral artery. A microcatheter is placed through the guide catheter and used to deliver the coil-tipped retriever across the clot and is then pulled back to deploy the retriever around the clot. The microcatheter and retriever are then pulled back, with the goal of pulling the clot, into the balloon guide catheter while the balloon is inflated, and a syringe is connected to the balloon guide catheter to aspirate the guide catheter during clot retrieval. This device has had initially positive results as compared to thrombolytic therapy alone.

Other thrombectomy devices utilize expandable cages, baskets, or snares to capture and retrieve clots. Temporary stents, sometimes referred to as stentrievers or revascularization devices, are utilized to remove or retrieve clot as well as restore flow to the vessel. A series of devices using active laser or ultrasound energy to break up the clot have also been utilized. Other active energy devices have been used in conjunction with intra-arterial thrombolytic infusion to accelerate the dissolution of the thrombus. Many of these devices are used in conjunction with aspiration to aid in the removal of the clot and reduce the risk of emboli. Suctioning of the clot has also been used with single-lumen catheters and syringes or aspiration pumps, with or without adjunct disruption of the clot. Devices which apply powered fluid vortices in combination with suction have been utilized to improve the efficacy of this method of thrombectomy. Finally, balloons or stents have been used to create a patent lumen through the clot when clot removal or dissolution was not possible.

Notwithstanding the foregoing, there remains a need for new devices and methods for treating vasculature occlusions in the body, including acute ischemic stroke and occlusive cerebrovascular disease. In particular, as will be discussed in more detail below, because of the variation in levels of tortuosity and the large variability of certain segments of the intracranial carotid artery (e.g., the petrous-cavernous path) in stroke patients, there is a need for an anatomy matched catheter design.

### SUMMARY

In accordance with one aspect, there is provided a neurovascular catheter for insertion into an internal carotid artery of a patient. In some embodiments, the neurovascular catheter can include an elongate flexible body and a flexible distal portion. The elongate flexible body can include a length of less than about 110 cm and can include the flexible distal portion. The flexible distal portion can include a length of between about 10 cm and about 18 cm. The flexible distal portion can include a distal tip, a transitional portion, and a support portion. In some embodiments, the distal tip is configured to be positioned within a cavernous segment of the internal carotid artery. In some embodiments, the transitional portion proximal to the distal tip is less flexible than the distal tip, wherein the transitional portion is configured to be positioned within a petrous segment of the internal carotid artery. In some embodiments, the support portion proximal to the transitional portion is less flexible than the transitional portion. In some embodiments, the support portion is configured to extend proximally to a base of the patient's skull.

In any of the embodiments described herein, the stiffness of a catheter portion is determined using a cantilever beam test, such as a cantilever beam test with a 5 mm gage length and 4 mm displacement to determine a peak load value.

In some embodiments, the elongate flexible body has a length of between about 98 cm and about 102 cm. In some embodiments, the flexible distal portion has a length of between about 12 cm and about 16 cm. In some embodiments, the distal tip has a length of between about 15 mm to about 20 mm. In some embodiments, the transitional portion has a length of between about 1.0 cm and 3.5 cm. In some embodiments, the proximal end of the transitional portion is positioned a distance of between about 5.5 cm and about 7.5 cm from a distal end of the flexible distal portion. In some embodiments, the distal tip comprises a constant stiffness along a length of the distal tip. In some embodiments, the stiffness is between about 20 gF to about 30 gF, wherein the stiffness is determined using a cantilever beam test with a 5 mm gage length and 4 mm displacement to determine a peak load value. In some embodiments, the flexible distal portion further comprises a flexible portion that extends proximal to the distal tip and comprises a stiffness that increases along a length of the flexible portion. In some embodiments, the flexible portion has a first stiffness at a first end of the flexible portion that is between about 20 gF to about 30 gF, and wherein the flexible portion has a second stiffness at a second end of the flexible portion that is between about 60 gF to about 65 gF. In some embodiments, the transitional portion comprises a constant stiffness along a length of the distal tip. In some embodiments, the stiffness is between about 50 gF to about 70 gF. In some embodiments, the support portion comprises a stiffness that increases along a length of the support portion. In some embodiments, the support portion has a first stiffness at a first end of the support portion that is between about 60 gF to about 70 gF, and wherein the support portion has a second stiffness at a second end of the support portion that is at least 400 gF.

In accordance with another aspect, disclosed is a neurovascular catheter for insertion into an internal carotid artery of a patient. The neurovascular catheter can include an elongate body comprising a flexible distal segment. In some embodiments, the flexible distal segment comprises a length of between about 10 cm and about 18 cm. In some embodiments, the elongate flexible body comprises a length of less than about 110 cm. The flexible distal segment can include a distal portion, a transitional portion, and a support portion. The distal portion can include a tracking tip and an increasing stiffness portion. In some embodiments, the distal portion has a length between about 20 mm and 40 mm. The tracking tip can be configured to be positioned within a cavernous segment of the internal carotid artery. In some embodiments, the tracking tip has a length between about 10 mm and about 20 mm. The increasing stiffness portion can be proximal to the tracking tip. In some embodiments, the increasing stiffness portion has a length between about 5 mm and about 15 mm. The transitional portion can be proximal to the distal portion that is less flexible than the tracking tip, wherein the transitional portion is configured to be positioned within a petrous segment of the internal carotid artery, wherein the transitional portion has a length of between about 30 mm and about 40 mm. In some embodiments, the support portion is proximal to the transitional portion and is less flexible than the transitional portion. In some embodiments, the support portion is configured to extend proximal to a base of a patient's skull, wherein the support portion comprises a stiffness that increases proximally along a length of the support portion, wherein the support portion has a length between about 70 mm and about 80 mm. The flexible distal segment can have a flexibility profile that is measurable with a cantilever beam test with a 5 mm gage length and 4 mm displacement to determine a peak load value. In some embodiments, the peak load value in the tracking tip is less than about 30 gF. In some embodiments, the peak load value in the increasing stiffness portion increases from between about 20 gF and about 30 gF to between about 50 gF and about 70 gF over the length of the increasing stiffness portion. In some embodiments, the peak load value in the transitional portion is between about 50 gF and about 70 gF. In some embodiments, the peak load value at a first end of the support portion is between about 50 gF and about 70 gF and at a second end of the support portion is at least about 400 gF.

In some embodiments, the elongate flexible body has a length of about 100 cm. In some embodiments, the flexible distal segment has a length of about 14 cm. In some embodiments, the distal portion has a length of about 30 mm. In some embodiments, the tracking tip has a length of about 17 mm. In some embodiments, the increasing stiffness portion has a length of about 13 mm. In some embodiments, the transitional portion has a length of about 35 mm. In some embodiments, the support portion has a length of about 75 mm.

In another aspect, disclosed is a neurovascular catheter for insertion into an internal carotid artery of a patient. In some embodiments, the neurovascular catheter includes a flexible distal segment for insertion into an internal carotid artery of a patient. The flexible distal segment can include a distal portion, a transitional portion, and a support portion. In some embodiments, the distal portion includes a tracking tip and an increasing stiffness portion. The tracking tip can be configured to be positioned within a cavernous segment of the internal carotid artery. In some embodiments, the transitional portion is configured to be positioned within a petrous segment of the internal carotid artery. The support portion can be configured to extend proximally to a base of a patient's skull. In some embodiments, the flexible distal segment can have a flexibility profile that is measurable with a cantilever beam test with a 5 mm gage length and 4 mm displacement to determine a peak load value. In some embodiments, the peak load value in the tracking tip is less than about 30 gF. In some embodiments, the peak load value in the increasing stiffness portion increases from between about 20 gF and about 30 gF to between about 50 gF and about 70 gF over a length of the increasing stiffness portion. In some embodiments, the peak load value in the transitional portion is between about 50 gF and about 70 gF. In some embodiments, the peak load value at a first end of the support portion is between about 50 gF and about 70 gF and at a second end of the support portion is at least about 400 gF.

In some embodiments, the flexible distal segment has a length of between about 10 cm and about 18 cm. In some embodiments, the distal portion has a length between about 20 mm and 40 mm. In some embodiments, the tracking tip has a length between about 10 mm and about 20 mm. In some embodiments, the increasing stiffness portion has a length between about 5 mm and about 15 mm. In some embodiments, the transitional portion has a length of between about 30 mm and about 40 mm. In some embodiments, the support portion has a length between about 70 mm and about 80 mm.

In another aspect, disclosed is a method for accessing a petrous-cavernous segment of a patient. The method can include providing a catheter comprising a flexible distal segment comprising a distal portion having a tracking tip and an increasing stiffness portion, a transitional portion, and a support portion. In some embodiments, the method includes advancing the flexible distal segment of the catheter into an internal carotid artery of a patient such that. The tracking tip of the flexible distal segment can be positioned within a cavernous segment of the internal carotid artery. The transitional portion can be positioned within a petrous segment of the internal carotid artery. The support portion can extend to a base of a patient's skull.

In some embodiments, the tracking tip of the flexible distal segment of the catheter is inserted until a tactile feedback is received at a proximal end of the catheter indicating that the transitional portion has engaged with an anatomy of the petrous segment. In some embodiments, the catheter has a length of less than 110 cm. In some embodiments, the catheter has a length of 100 cm. In some embodiments, the flexible distal segment has a length of between 10 cm and 18 cm. In some embodiments, the flexible distal segment has a length of 14 cm. In some embodiments, the distal portion has a length between about 20 mm and 40 mm. In some embodiments, the distal portion has a length of about 30 mm. In some embodiments, the tracking tip has a length between about 10 mm and about 20 mm. In some embodiments, the tracking tip has a length of about 17 mm. In some embodiments, the increasing stiffness portion has a length between about 5 mm and about 15 mm. In some embodiments, the increasing stiffness portion has a length of about 13 mm. In some embodiments, the transitional portion has a length of between about 30 mm and about 40 mm. In some embodiments, the transitional portion has a length of about 35 mm. In some embodiments, the support portion has a length between about 70 mm and about 80 mm. In some embodiments, the support portion has a length of about 75 mm. In some embodiments, the distal tip is softer than the transitional portion, and wherein the transitional portion is softer than the support portion. In some embodiments, the method is configured to treat a tandem lesion.

In some embodiments, the flexible distal segment of the method can include a flexibility profile that is measurable with a cantilever beam test with a 5 mm gage length and 4 mm displacement to determine a peak load value. In some embodiments, the peak load value in the tracking tip is less than about 30 gF. In some embodiments, the peak load value in the increasing stiffness portion increases from between about 20 gF and about 30 gF to between about 50 gF and about 70 gF over a length of the increasing stiffness portion. In some embodiments, the peak load value in the transitional portion is between about 50 gF and about 70 gF. In some embodiments, the peak load value at a first end of the support portion is between about 50 gF and about 70 gF and at a second end of the support portion is at least about 400 gF.

Any feature, structure, or step disclosed herein can be replaced with or combined with any other feature, structure, or step disclosed herein, or omitted. Further, for purposes of summarizing the disclosure, certain aspects, advantages, and features of the embodiments have been described herein. It is to be understood that not necessarily any or all such advantages are achieved in accordance with any particular embodiment disclosed herein. No individual aspects of this disclosure are essential or indispensable. Further features and advantages of the embodiments will become apparent to those of skill in the art in view of the Detailed Description which follows when considered together with the attached drawings and claims.

### BRIEF DESCRIPTION OF THE DRAWINGS

FIGS. 1A-IF depict an alternative sequence of steps involved in accessing a neurovascular occlusion for aspiration.

FIG. 2 illustrates a schematic of the internal carotid artery.

FIG. 3A illustrates anatomical photos of the carotid siphon and the variability in the severity of tortuosity that can be present.

FIG. 3B illustrates anatomical photos of the posterior vertebral arteries and the variability in the severity of tortuosity that can be present.

FIG. 3C illustrates anatomical photos of the cavernous segment of the internal carotid artery and the variability in the severity of tortuosity that can be present.

FIG. 3D illustrates anatomical photos of the cervical segment of the internal carotid artery and the variability in the severity of tortuosity that can be present.

FIG. 4A illustrates a catheter intended for higher placement within the brain when it has a distal placement in the M1 segment of the middle cerebral artery.

FIG. 4B illustrates a catheter intended for higher placement within the brain when it has a low placement in the petrous segment of the internal carotid artery.

FIG. 4C illustrates a catheter intended for lower placement within the brain when it has a low placement in the petrous segment of the internal carotid artery.

FIG. 5A illustrates an embodiment of a distal end of the presently disclosed catheter.

FIG. 5B illustrates an embodiments of a tracking tip of the distal end of the disclosed catheter of FIG. 5A.

FIGS. 6A-6B illustrates a schematic describing the flexibility profile that may be desirable for the distal segment of the catheter.

FIG. 7 depicts a graph of modulus or durometer of the catheter along the length of the catheter, from the proximal end to the distal end.

FIG. 8 illustrates a tandem lesion positioned in the external carotid artery.

FIGS. 9A and 9B compare a catheter having a longer distal flexible segment with a catheter having a shorter distal flexible segment.

### DETAILED DESCRIPTION

### Overview

Although primarily described in the context of neurovascular aspiration catheters with a single central lumen, the presently disclosed catheter can readily be modified to incorporate additional structures, such as permanent or removable column strength enhancing mandrels, two or more lumens such as to permit drug, contrast or irrigant infusion or to supply inflation media to an inflatable balloon carried by the catheter, or combinations of these features, as will be readily apparent to one of skill in the art in view of the disclosure herein. In addition, embodiments of the presently disclosed catheter will be described primarily in the context of removing obstructive material from remote vasculature in the brain but has applicability as an access catheter for delivery and removal of any of a variety of diagnostics or therapeutic devices with or without aspiration.

The catheters disclosed herein may readily be adapted for use throughout the body. For example, embodiments of the presently disclosed catheter shafts may be dimensioned for use throughout the coronary and peripheral vasculature, the gastrointestinal tract, the urethra, ureters, Fallopian tubes and other lumens and potential lumens, as well. The catheters presently disclosed herein may also be used to provide minimally invasive percutaneous tissue access, such as for diagnostic or therapeutic access to a solid tissue target (e.g., breast or liver or brain biopsy or tissue excision), delivery of laparoscopic tools or access to bones such as the spine for delivery of screws, bone cement or other tools or implants. Examples of such catheters are illustrated in, for example, U.S. Pat. No. 10,183,145 to Yang, et al., and U.S. Pat. No. 10,835,272 to Yang, et al. the disclosure of which are incorporated in its entirety herein by reference.

### Overview of Method of Treatment

### Treatment of Occlusion in Internal Carotid Artery

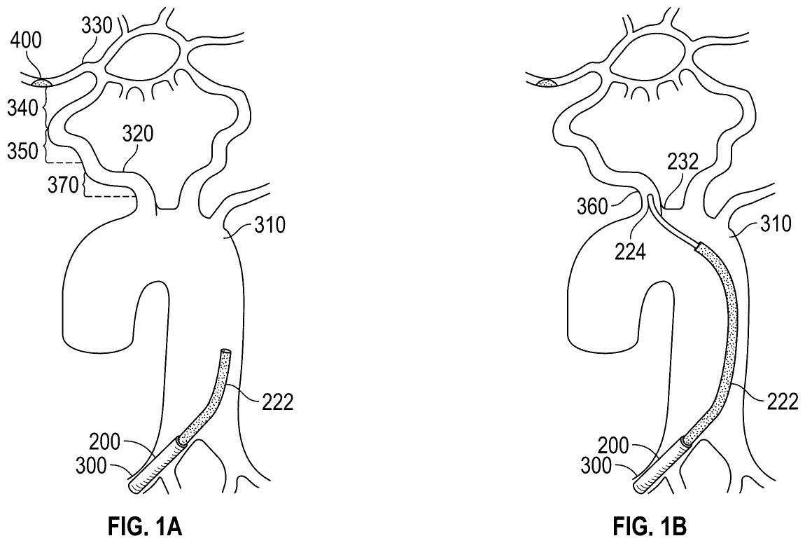

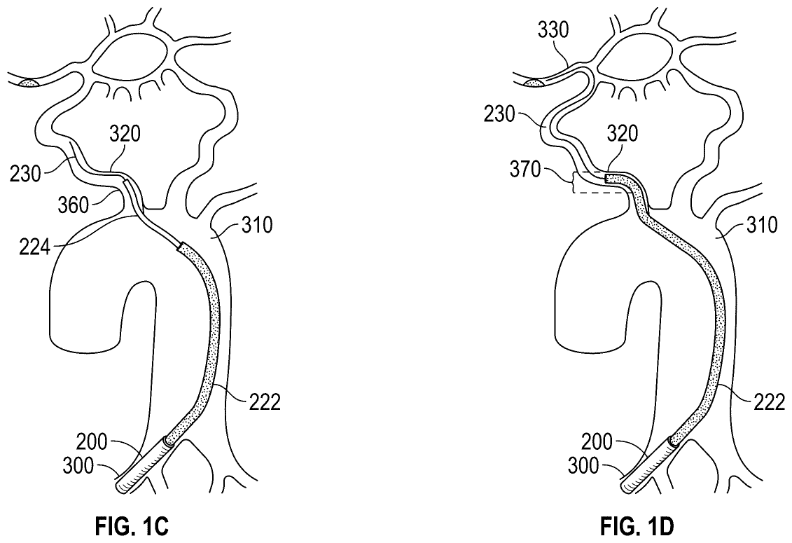

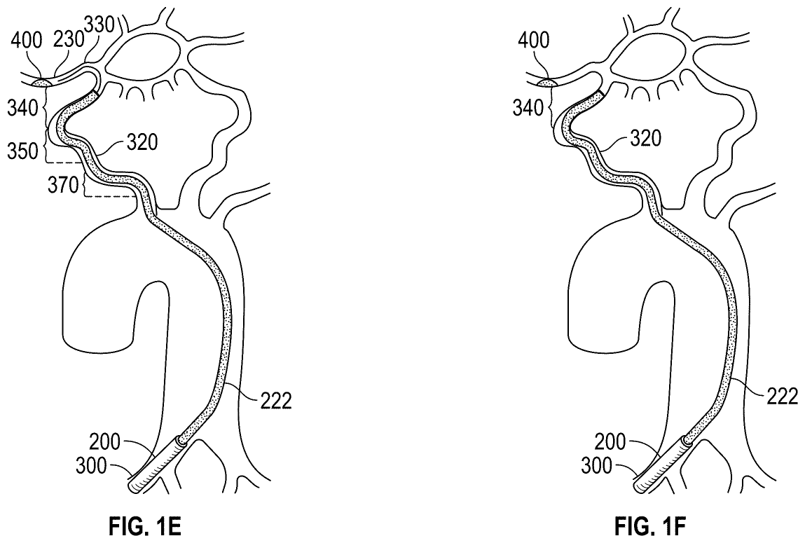

FIGS. 1A-1F illustrate a method for aspirating a thrombotic occlusion. The method for aspirating a thrombotic occlusion can use a transitional guidewire and a transitional guide sheath. The transitional guidewire has a soft and trackable distal segment with a smaller diameter so that the transitional guidewire may be advanced deeper into the neurovasculature. In some embodiments, using a transitional guidewire and a transitional guide sheath that can be advanced to an area near the clot eliminates the need to use a second guidewire or a reperfusion catheter to reach the clot.

Referring to FIG. 1A, an introducer sheath 200 is introduced at femoral artery 300. The outer diameter of the introducer sheath 200 may be equal to or less than about 12F, 11F, 10F, 9F, 8F, 7F, or 6F. Then, a transitional guide sheath 222 such as the combination access and aspiration catheter discussed in greater detail below is inserted through the introducer sheath 200 at the femoral artery 300. The outer diameter of the guide sheath 222 may be equal to or less than about 9F, 8F, 7F, 6F, 5F, 4F, or 3F. Referring to FIG. 1B, an insert catheter 224 is inserted through the transitional guide sheath 222. The outer diameter of the insert catheter 224 may be less than about 9F, 8F, 7F, 6F, 5F, 4F, or 3F, and the inner diameter of the transitional guide sheath 222 may be greater than the outer diameter of the insert catheter 224. In some cases, a first guidewire may be introduced through the insert catheter 224 (not shown in FIG. 1B). The diameter of the proximal section of the first guidewire may be equal to or less than about 0.079″, about 0.066″, about 0.053″, about 0.038″, about 0.035″, about 0.030″, or about 0.013″.

The transitional guide sheath 222, the insert catheter 224, and optionally the first guidewire is tracked up to the aortic arch 310. See FIG. 1B. The insert catheter 224 may be used to select the origin of a vessel. In FIG. 1B, the insert catheter 224 engages the origin 1216 of the brachiocephalic artery 360. An angiographic run may be performed by injecting contrast media through the insert catheter 224. In the cases where the first guidewire is used before the angiographic run, the first guidewire is preferably removed prior to injecting the contrast media.

Referring to FIG. 1C, the transitional guidewire 230 is inserted through the lumen of the insert catheter 224 or guide sheath 222. The diameter of at least a portion of the transitional guidewire 230 (e.g., proximal diameter) is substantially similar to that of the first guidewire 232. The diameter of at least a portion of the transitional guidewire 230 (e.g., distal diameter) may be smaller than that of the first guidewire 232 and may have a diameter along a proximal segment of at least about 0.030″ and in one implementation about 0.038″. A transition begins within the range of from about 15 cm-30 cm from the distal end, and typically no more than about 20 cm or 25 cm from the distal end, distally of which the diameter tapers down to no more than about 0.018″ and in one implementation about 0.016″.

Referring to FIG. 1D, if utilized, the insert catheter 224 may be removed because it is too stiff to be advanced to the MCA 330. In some embodiments, the transitional guidewire 230 provides sufficient back up support that the combination access and aspiration catheter 224 may be advanced directly over the transitional guidewire without any intervening devices. Then, the transitional guidewire 230 is advanced to the MCA 330. The transitional guidewire 230 may have a distal segment that has a smaller diameter than that of a guidewire 1126 described in similar systems. The distal segment of the transitional guidewire 230 comprises a soft and atraumatic tip and can be tracked to the remote neurovasculature such as the MCA 330, which is distal to the petrous segment 370 of the ICA 320.

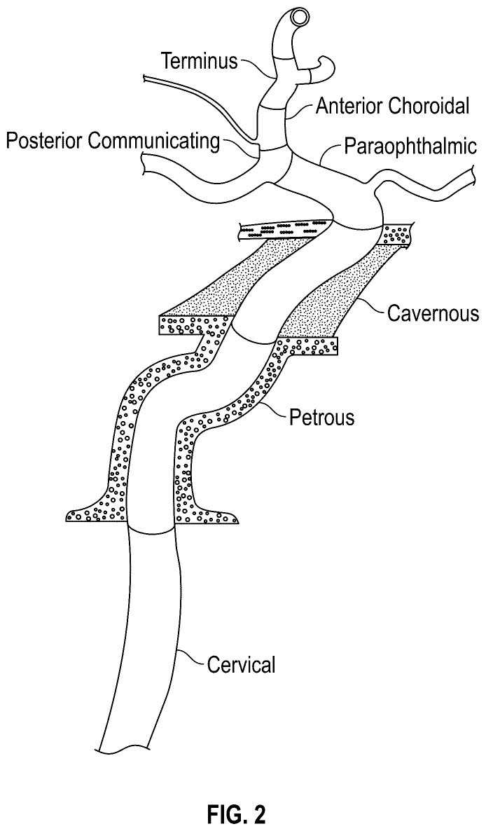

Referring to FIG. 1E, the transitional guide sheath 222 is advanced to or beyond the cavernous segment 350 or the cerebral segment 340 of the ICA 320. In some embodiments, the transitional guide sheath 222 may be advanced to the cavernous segment 350 or the cerebral 340 segment of the ICA 320 beyond the petrous segment 370 because the transitional guide sheath 222 has a soft yet trackable distal segment described in further detail below, for example in connection with FIG. 2. The larger proximal diameter and/or stiffer body of the transitional guidewire 230 may provide better support for the transitional guide sheath 222 to track through the vasculature.

Referring to FIG. 1F, after the transitional guide sheath 222 is advanced to the cerebral segment 340 of the ICA 320, the transitional guidewire 230 is removed. Then, a vacuum pressure is applied at the proximal end of the transitional guide sheath 222 to aspirate the clot 400 through the central lumen of the transitional guide sheath 222. The inner diameter of the transitional guide sheath 222 may be equal to about or greater than about 0.100″, about 0.088″, about 0.080″, about 0.070″, or about 0.060″. The inner diameter of the transitional guide sheath 222 is larger than the aspiration catheter 1128, which translates to more effective aspiration. The cross-sectional area of the central lumen of the transitional guide sheath 222 may be almost twice as large as that of the largest aspiration catheter 1128 currently available.

If the guide sheath 222 is not able to track deep enough into the distal vasculature to reach the clot or other desired target site, a telescopic extension segment as discussed elsewhere herein may be introduced into the proximal end of sheath 222 and advanced distally to extend beyond the distal end of the sheath 222 and thereby extend the reach of the aspiration system. In some embodiments, the extension segment has an ID of about 0.070″. Alternatively, the ID of the insert catheter may be greater than the OD of the guidewire by a sufficient amount to provide a contrast injection lumen between the guidewire and the insert catheter, enabling introduction of contrast without the need to remove the guidewire.

If thrombotic material is not able to be drawn into the sheath 222 or extension segment under constant vacuum, pulsatile vacuum may be applied. If pulsatile vacuum does not satisfactorily capture the clot, an agitator may be advanced through the sheath 222 and extension segment to facilitate drawing the clot into the central lumen. Alternatively, the transition may be a step down in flexibility but with a constant outside diameter throughout,

### Overview of Intracranial Catheters

In some embodiments, intracranial anatomy can be reached with pre-shaped insert catheters. These insert catheters can be configured to help guide catheters to reach a target site in the intracranial anatomy on the initial ascent. Subsequently, aspiration catheters can be used to reach an occlusion (e.g., a clot face) and remove it from the body. Generally, about 86% of clots are positioned at the M1 segment of the middle cerebral artery (MCA). However, depending on the position of the clot, the aspiration catheter can frequently have insufficient support to reach the site of the clot. In some embodiments, an occlusion can be positioned within an internal carotid artery.

FIG. 2 illustrates a schematic and an anatomic view of the internal carotid artery. The internal carotid artery can have great variability in length and in tortuosity. In particular, there exist tight radius turns in the treatment pathway that are most commonly found in the carotid siphon, vertebral (posterior), petrous/cavernous, cervical, and branchiocephalic (arch).

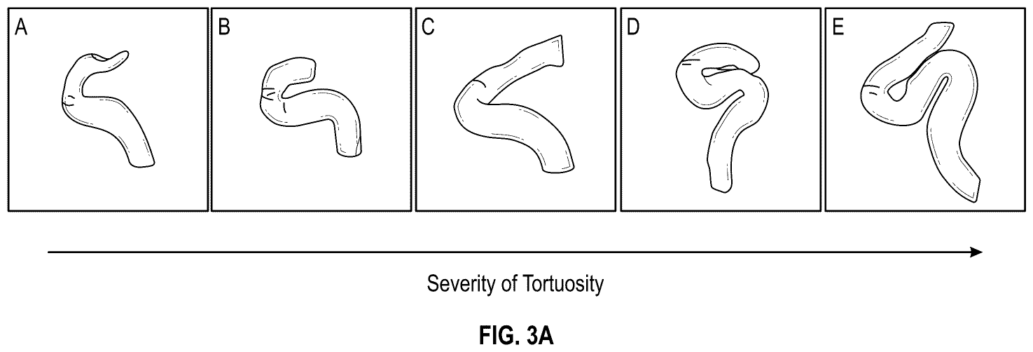

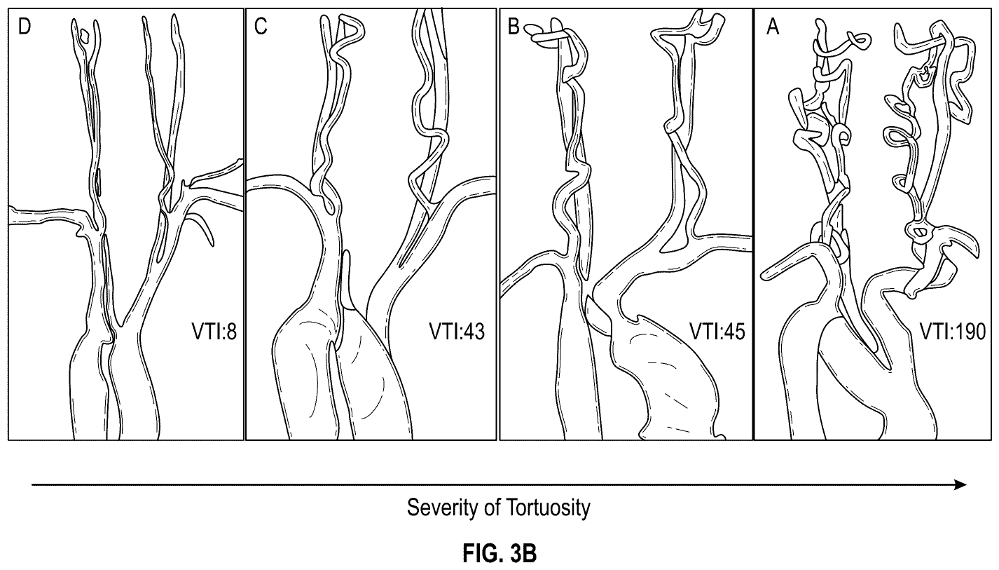

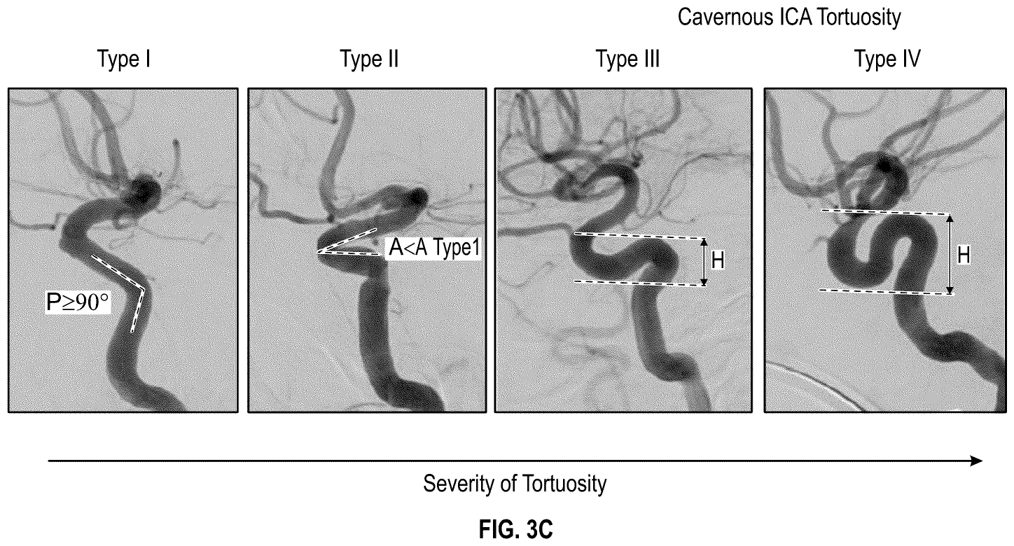

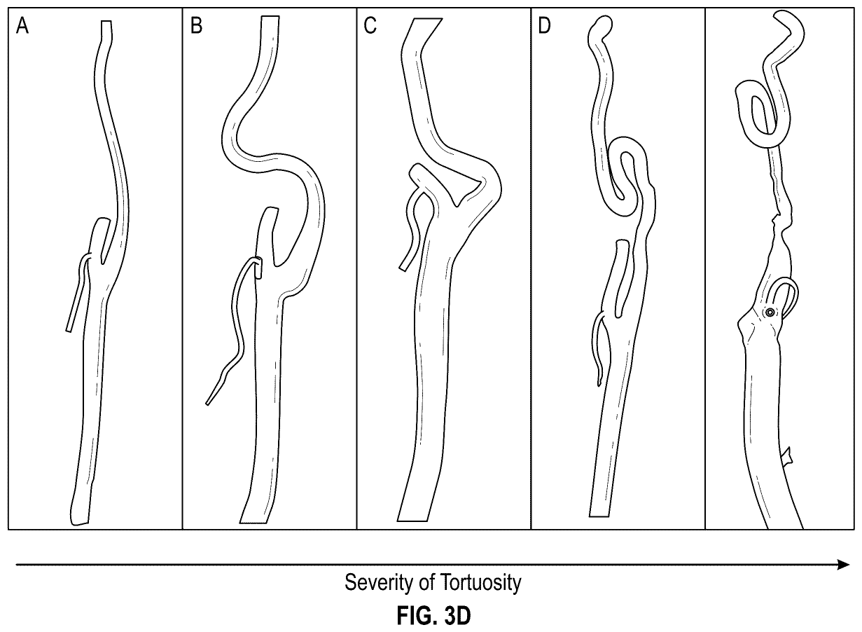

FIGS. 3A-3D illustrate an example of the range and variability of tortuosity that can be present in various portions of the internal carotid artery. FIG. 3A illustrates a range of the severity of tortuosity that can be present in the carotid siphon. FIG. 3B illustrates a range of the severity of tortuosity that can be present in the posterior vertebral arteries. FIG. 3C illustrates a range of the severity of tortuosity that can be present in the cavernous segment. FIG. 3D illustrates a range of the severity of tortuosity that can be present in the cervical segment. Depending on the severity of tortuosity and the length to be reached, different material and/or flexibility profiles of the catheter are required as there exists a tradeoff between the navigability/flexibility of a softer material and the force transmission provided by a harder material.

Another consideration in the development of an intracranial catheter is the length of particular segments of the internal carotid artery. As an example, there is great variation in the petrous-cavernous segment of the internal carotid artery in stroke patients. In particular, a stroke patient can therefore have a petrous-cavernous segment with approximately 6.2 cm in variation. This variation is significantly greater than what is found in the general population. Because of the large variation of petrous to cavernous path lengths, a single catheter design may be unable to meet the needs of all patients.

An anatomy matched catheter design may therefore be desirable to provide for varying levels of tortuosity and the large variability of petrous-cavernous path length in patients. Furthermore, the catheter may be designed to have an appropriate length and flexibility profile to not only navigate the tortuosity of the artery, but also to provide a physician with sufficient support and force along the length of the distal end of the catheter so as to advance the distal tip of the catheter to a target location. In some embodiments, the presently disclosed catheter is designed for stroke patients with a petrous-cavernous segment that is average or less than average length.

### Enhanced Control & Improved Efficiency of Procedure

In some embodiments, embodiments of the disclosed catheter can have a shorter effective length than existing catheters to provide for a more neutral arm positioning during operation. In some embodiments, the shorter effective length of the catheter from the distal hub can improve the ergonomics of hand placement and the efficiency of the procedure. The shorter length of the catheter can reduce the physician's reach and improve the ergonomics of movement from the introducer sheath to the hub during the procedure. In some embodiments, the effective length of the catheter is about 100 cm, between about 98 cm to about 102 cm, between about 96 cm to about 104 cm, between about 94 cm to about 106 cm, between about 92 cm to about 108 cm, between about 90 cm to about 110 cm, between about 88 cm to about 112 cm, between about 86 cm to about 114 cm, between 84 cm to about 116 cm, between about 82 cm to about 118 cm, between about 80 cm to about 120 cm, and any value in between the ranges listed, including endpoints. The provided lengths of the catheters are compatible with commercially available adjunctive devices (e.g., insert catheters, carotid stent delivery systems).

### Anatomically Driven Design

Tortuous vasculature is a common reason for failure to treat vasculature occlusions in the body due to inability to track the catheter to the location of the disease. Navigating catheters through tortuous anatomy such as neurovasculature can be a challenge. The catheter has to be very soft so as not to damage the vessel wall. At the same time, it also has to be able to negotiate multiple tight turns without kinking. In addition, it has to have sufficient column strength to transmit force axially for advancing through the vasculature. All these performance characteristics are competing design requirements. It is difficult to optimize one performance characteristic without sacrificing the others. In some embodiments, the flexible distal end of the presently disclosed catheter may comprise a multi-layer construct having a high degree of flexibility and sufficient push ability to reach deep into the cerebral vasculature, such as at least as deep as the distal cavernous.

Embodiments of the presently disclosed catheter are designed to fit a specific anatomical need. In particular, the aspiration catheter is designed to navigate to the distal cavernous segment consistently. In some embodiments, the catheter can go to the M1 segment of the MCA in patients with low tortuosity. As discussed above, the catheter in some embodiments is designed to be used in patients with average or shorter than average petrous-cavernous segments. In some examples, the disclosed catheter is designed for lower placement in the internal carotid artery (i.e., the petrous segment). In some embodiments, the disclosed catheter is configured to be used in neurovasculature that is less tortuous. In some embodiments, the length of the distal flexible segment of the catheter allows the catheter to treat a tandem lesion as the length of the flexible distal segment which corresponds generally with the distance between the ECA bifurcation and the arch.

### Low Positioned Catheter

The distal tip of the catheter in some embodiments can be positioned anywhere intracranially along the length of the internal carotid artery. Many existing catheters are configured to allow the distal tip of the catheter to be placed in a high position within the brain. For example, the distal tip can be positioned in the M1 of the middle cerebral artery which is distal to the internal carotid artery (ICA). There can be many advantages with positioning a catheter above the ICA in the brain. For example, the further intracranially a catheter is positioned, the more functional the device can be within the brain. However, many physicians are uncomfortable with advancing the catheter so far intracranially as complications and possible rupture of the artery can occur during the procedure. There is therefore a need for a catheter that can be advanced to a lower position within the brain (i.e., the vertical portion of the petrous segment of the internal carotid artery) while providing sufficient support for subsequently advancing an aspiration catheter.

The presently disclosed catheter in some embodiments has material properties that are designed to allow the catheter to be placed in a lower position (e.g., the petrous segment) while providing sufficient support to an aspiration catheter advanced along its length.

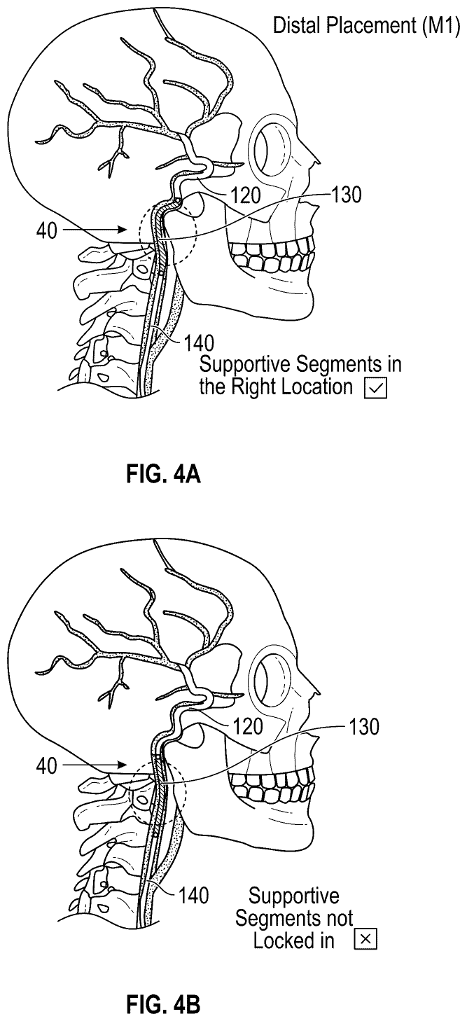

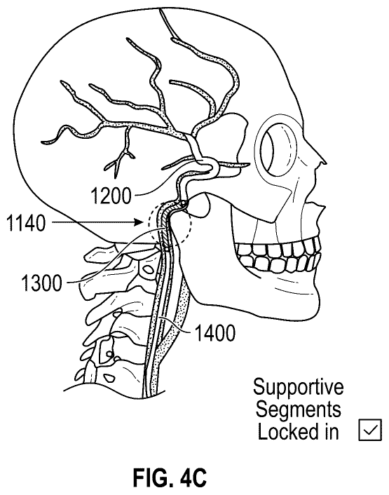

Because catheters are designed to fit specific anatomical needs, a catheter intended to be positioned further intracranially in the brain (e.g., distal to the horizontal portion of the petrous segment) will frequently be unsuitable for positioning in a lower portion of the artery (e.g., proximal portion of the petrous segment, such as the vertical portion of the petrous segment). As an example, FIG. 4A illustrates the use of a catheter 100 designed for higher placement in the brain when it is placed in the M1 segment of the middle cerebral artery. The transition segment 110 between the proximal stiffer segment 120 and the adjacent distal more flexible segment 130 lodges in the tortuous petrous segment vasculature at the base of the skull, stabilizing the catheter in place. As shown, the higher stiffness of the catheter proximal to the transition segment 110 of this higher placement catheter “locks” 40 into the artery and will provide support when an aspiration catheter is advanced therethrough.

In contrast, FIG. 4B illustrates a catheter 100 designed for higher placement when it instead has a lower placement in the internal carotid artery. As illustrated, the transition segment 110 is placed well below the base of the skull so the distal end of the stiffer proximal segment 120 is not able to engage the tortuosity and form a soft lock. The soft distal segment 130 extends proximally from the base of the skull, placing the transition too far proximally of the base of the skull to form the soft lock 40. This soft distal segment 130 will absorb force transmission and fail to sufficiently support an aspiration catheter as it is advanced distally.

FIG. 4C illustrates a catheter that is designed for lower placement in the brain (i.e., the petrous of the internal carotid artery). The transition segment 1110 and distal end 1122 of the proximal segment 1120 are engaged in the vessel to help the catheter stay in place and provide support as an aspiration catheter is advanced therethrough. The transition segment 1110 between catheter segments having dissimilar material properties is preferably placed spaced apart from the distal end of the catheter by an amount equal to the distance between the soft lock 1140 and the intended final location of the catheter distal tip. This allows a physician to receive tactile feedback of the soft lock in every case, regardless of the final position of the distal catheter tip, by selecting a catheter designed to reach that specific target site. The design of the catheter thus preferably complements the anatomy in which it is used.

### Overview of Anatomically Matched Catheter

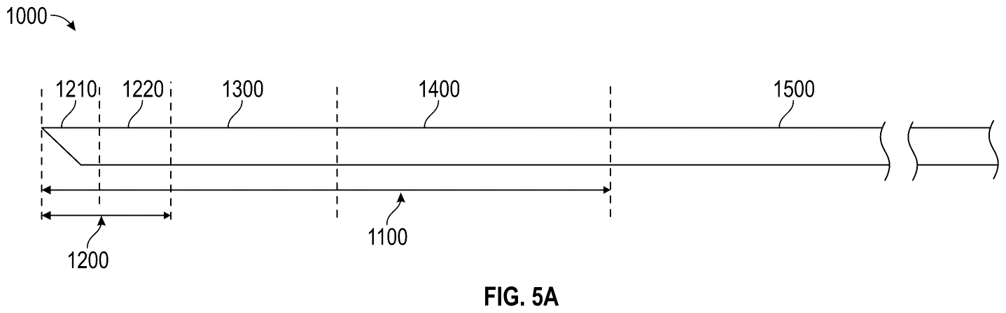

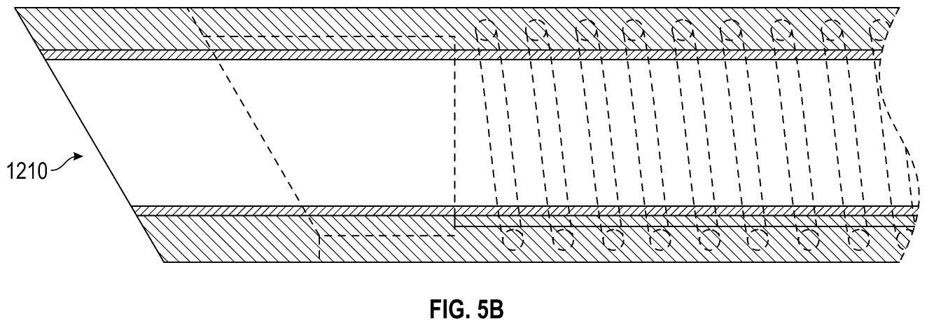

FIG. 5A illustrates an example of an anatomically matched catheter 1000 for lower placement within the brain. The catheter 1000 is designed to consistently navigate the distal cavernous segment. The catheter 1000 is designed in some embodiments for use on patients with average or shorter than average petrous-cavernous segments and/or in patients with neurovasculature that is less tortuous. As will be discussed in more detail below, the distal segment 1100 of the catheter 1000 is designed in some embodiments for placement in the petrous segment of the internal carotid artery.

The catheter 1000 can include a distal segment 1100 and a proximal segment 1500. The distal segment 1100 can include a distal portion 1200, a transitional portion 1300, and a support portion 1400. The distal segment 1100 can increase in flexibility as it extends distally from the support portion 1400 to the end of the distal portion 1200. In some embodiments, the distal segment 1100 can have a length of about 14 cm, between about 13 cm to about 15 cm, between about 12 cm to about 16 cm, between about 11 cm to about 17 cm, between about 10 cm to about 18 cm, and any value in between the ranges listed, including endpoints. In some examples, the catheter has a graduated flexibility profile through a plurality of transitions between axially adjacent sidewall segments having successively decreasing durometers in the distal direction. As will be discussed in more detail below, the catheter 1000 can provide sufficient pushability and support such that the insertion catheter remains stable during catheter delivery and advancement.

In some embodiments, in order to reduce the physician's reach and improve the ergonomics of movement from the introducer sheath to the hub during the procedure, the catheter 1000 can have an overall length of about 100 cm, between about 98 cm to about 102 cm, between about 96 cm to about 104 cm, between about 94 cm to about 106 cm, between about 92 cm to about 108 cm, between about 90 cm to about 110 cm, and any value in between the ranges listed, including endpoints.

### Distal Portion & Flexible Distal Tip of Distal Segment

In some embodiments, the distal portion 1200 can form the distal-most portion of the distal segment 1100 of the catheter 1000. The distal portion 1200 comprises enough flexibility to reach the intracranial vasculature above the skull base during access. The distal portion 1200 can comprise a tracking tip 1210 and an increasing stiffness portion 1220 that is proximal to the tracking tip 1210.

In some embodiments, the distal portion 1200 can have a length that is between about 0 cm to about 6 cm, between about 1 cm to about 5 cm, between about 2 cm to about 4 cm, or about 3 cm and any value in between the ranges listed, including endpoints. In some embodiments, the distal portion 1200 can comprise a material such as urethane.

The tracking tip 1210 can form the distal-most segment of the distal portion 1200 and the catheter 1000. The tracking tip 1210 can lead the catheter 1000 and can guide the catheter into the turns as the catheter 1000 is advanced. In some embodiments, the tracking tip 1210 can form the softest portion of the catheter and leads the catheter and buckles and turns the catheter 1000 when it hits the artery wall. The tracking tip 1210 of the catheter can be softer than the rest of the distal segment 1100 and the catheter 1000 in order to improve the catheter's ability to navigate tortuous intracranial vessels and follow the tortuous path to reach a target location.

In some embodiments, the tracking tip 1210 comprises a stretched or softened layer. This layer may first be formed by dip coating a mandrel (not shown) to provide a thin-walled tubular inside a layer of the catheter body. The dip coating may be produced by coating a wire such as a silver coated copper wire in PTFE, expanded-PTFE (e-PTFE), thermoplastic polyurethane (e.g., inherently hydrophilic, lubricious inner diameter property, low durometer), Fluorinated Ethylene Propylene (FEP), Polyvinylidene Fluoride (PVDF), or like material. The mandrel may thereafter be axially elongated to reduce its diameter and thereafter removed to leave the tubular inner liner. The outside surface of this tubular inner liner may thereafter be coated with a soft tie layer such as polyurethane, to produce a layer having a thickness of no more than about 0.005 inches, and in some implementations approximately 0.001 inches.

In some embodiments, the tracking tip 1210 comprises layer is stretched or softened in order to enhance flexibility of at least that section of the catheter. In some examples, the softening may be accomplished by one or more of: stretching the inner liner, applying one or more holes (e.g., any embodiments of holes described elsewhere herein) to the inner liner, applying heat to the inner liner, chemically treating the inner liner, altering manufacturing parameters of the inner liner, etc.

In some embodiments, a range of stretch of at least a portion of the inner liner may be about 20% to about 150% elongation; about 20% to about 75% elongation; about 100% to about 150% elongation; about 50% to about 90% elongation; about 60% to about 80% elongation; about 70% to about 80% elongation; about 50% to about 100% elongation; 20% to about 90% elongation; etc. For example, the softened or stretched portion inner liner may have a thickness of about 0.0001 inches to about 0.001 inches; about 0.00005 inches to about 0.0005 inches; about 0.00025 inches to about 0.00075 inches; about 0.0004 inches to about 0.0006 inches, about 0.0003 inches to about 0.0007 inches; about 0.0004 inches to about 0.0008 inches, etc.

In some embodiments the tracking tip 1210 can comprise an angled distal tip. FIG. 5B illustrates an embodiment of the angled tracking tip 1210. To provide for catheter tracking, the tracking tip 1210 can include a marker band. The marker band can have at least one and optionally two or three or more axially extending slits throughout its length to enable radial expansion. The marker band may comprise any of a variety of radiopaque materials, such as a platinum/iridium alloy. In some embodiments, the tracking tip 1210 can have an axial length of about 17 mm, between about 16 mm to about 18 mm, between about 15 mm to about 19 mm, between about 15 mm to about 20 mm, between about 14 mm to about 20 mm, between about 13 mm to about 21 mm, between about 12 mm to about 22 mm, between about 11 mm to about 23 mm, between about 10 mm to about 24 mm, and any value in between the ranges listed, including endpoints.

### Transitional Portion of Distal Segment

The distal segment 1100 can include a transitional portion 1300 that is positioned on a proximal end of the distal portion 1200. In some embodiments, the transitional portion 1300 is located on the distal segment 1100 to anatomically matched to engage and form a soft lock in the petrous segment of the internal carotid artery. The soft lock can be configured to secure the flexible distal segment 1100 into the petrous segment in order to provide support as an aspiration catheter is advanced. In some embodiments, the transitional portion 1300 can have a length within the range of between about 0 cm to about 7 cm, between about 1 cm to about 6 cm, between about 1.0 cm to about 3.5 cm, between about 2 cm to about 5 cm, between about 3 cm to about 4 cm, or about 3.5 cm and any value in between the ranges listed including endpoints.

### Support Portion of Distal Segment

The distal segment 1100 can include a support portion 1400 that is positioned at a proximal end of the transitional portion 1300. In some embodiments, the support portion 1400 forms the proximal end of the distal segment 1100 and extends proximally from the skull base. The support portion 1400 can increase in stiffness in the proximal direction between the proximal end of the transitional portion 1300 and the distal end of the proximal segment 1500. The support portion 1400 can form the stiffest portion of the distal segment 1100. In some embodiments, the flexibility profile of the support portion 1400 provides sufficient pushability and support for the distal portion 1200 and transitional portion 1300 of the distal segment 1000 as the catheter 1000 is advanced into the cavernous segment of the internal carotid artery and the transitional portion 1300 forms a soft lock in the petrous segment of the intracranial carotid artery. In some embodiments, the support portion 1400 can have a length of about 7.5 cm, between about 7 cm to about 8 cm, between about 6.5 cm to about 8.5 cm, between about 6 cm to about 9 cm, between about 5.5 cm to about 10 cm, and any value in between the ranges listed including endpoints.

### Proximal Segment

In some embodiments, the proximal segment 1500 forms a proximal end of the catheter 1000. As shown in FIG. 5, the proximal segment 1500 can extend from the proximal end of the distal segment 1100. In some embodiments, the proximal segment 1500 comprises a stiffer material, such as nylon 12. In some embodiments, the proximal segment 1500 can have a length of about 86 cm, between about 80 cm and about 90 cm, between about 75 and 95 cm, between about 70 cm and about 100 cm, and any value in between the ranges listed including endpoints.

### Flexibility Profile of Distal Segment

### Overview

The distal shaft stiffness profile can be sufficiently flexible for easy intracranial access during the initial ascent. In some embodiments, the flexibility of the profile of the catheter comprises seamless transitions for optimal navigation around and along tortuous intracranial vessels. In some examples, the catheter comprises a rate of change of stiffness along the distal shaft that corresponds to the anatomical tortuosity for minimized shaft kinking, buckling force, and push force in order to improve support during both access and catheter delivery. The anatomically matched stiffness transition profile can also provide stability when delivering smaller catheters. Also, the transition profile ensures that the catheter does not move proximally and stays seated in a desired location when translating the smaller catheters. In some embodiments, the flexibility profile of the distal flexible segment provides excellent support when it is placed proximal to the siphon in a patient having average anatomy. In some embodiments, the flexibility profile of the catheter provides stability to the catheter when the physician wants to leave the device low and during the initial ascent.

The catheter 1000 is configured to be advanced through the internal carotid artery such that the tracking tip 1210 is positioned within the cavernous segment of the internal carotid artery. The flexibility profile of the distal segment 1100 is designed such that the change in flexibility on the proximal and distal ends of the transitional portion 1300 provides a tactile feedback to the physician when the transitional portion 1300 is seated in the petrous segment of the internal carotid artery (referred to herein as a “soft lock.”). The “soft lock” comes in the form of a mechanical tactile feedback to the physician where there is a slight pushback in the catheter to indicate that the transitional portion 1300 of the flexible distal segment 1200 has engaged with the anatomy of the petrous segment. In some examples, the support portion 1400 of the distal segment 1100 extends proximally from the skull base.

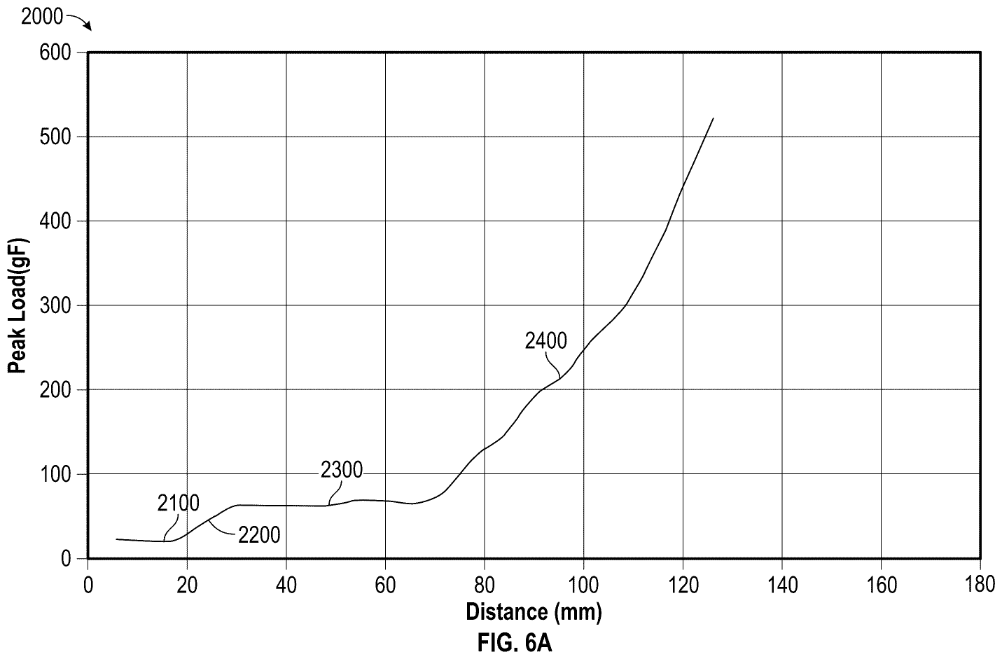

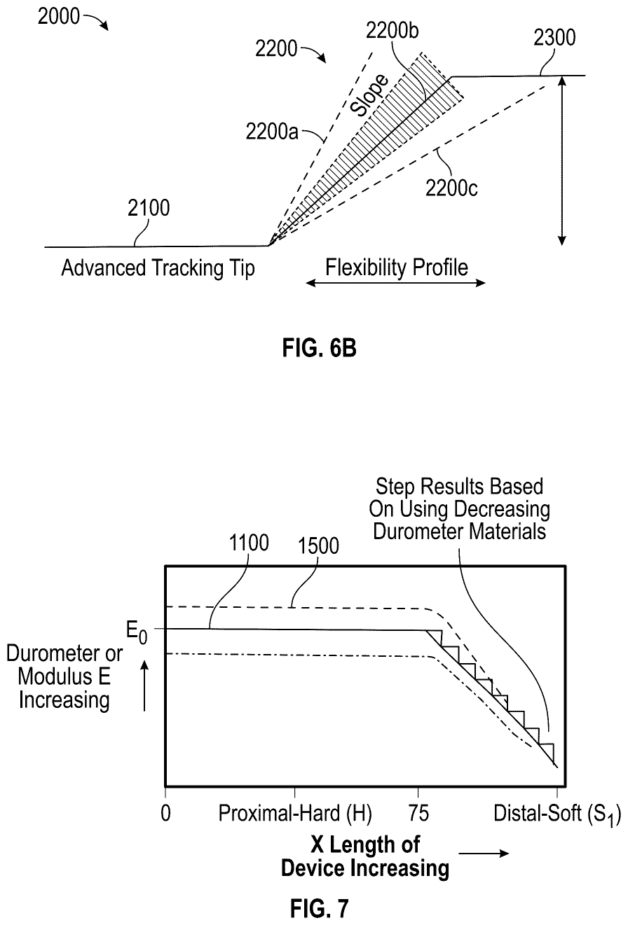

FIG. 6A illustrates the flexibility profile of the distal segment 1100 of the catheter 1000 as it extends (on the left) from the distal tracking tip 1210 to the support portion 1400 (on the right). In some embodiments, catheter flexibility can be determined using the cantilever beam test. The cantilever beam test can be done along the length of the catheter to characterize the local segment stiffness and the rate of stiffness change. The transitions and flexibility of intervening catheter shaft segments can be related to the anatomical tortuosity that the area of the catheter is designed to be navigating through. In the present application, the flexibility of the catheter 1000 was tested at 6 mm increments across the length of the distal portion 1200, starting from the distal end of the tracking tip 1210 to the proximal end of the support portion 1400. The catheter 1000 is secured at a 5 mm distance from the target location to be measured. This is the gage length (e.g., 5 mm) used by the cantilever beam test. A force is then applied to cause a 4 mm displacement at the target location of the catheter 1000, wherein the peak load is measured within the 4 mm displacement. The amount of force applied is measured at each sampling location (e.g., every 6 mm) to create a flexibility profile for the catheter 1000. The flexibility profile 2000 shown in FIG. 18 illustrates the peak load (gF) experienced at 6 mm increments along the length of the distal segment 1100. The flexibility profile 2000 includes section 2100, illustrating the peak load experienced by the tracking tip 1210 of the distal portion 1200; section 2200, illustrating the peak load experienced by the increasing stiffness portion 1220 of the distal portion 1200; section 2300, illustrating the peak load experienced by the transitional portion 1300; and section 2400, illustrating the peak load experienced by the support portion 1400.

Section 2100 illustrates the flexibility profile along the length of the tracking tip 1210. As discussed above, section 2100 represents the peak load along the length of the tracking tip 1210 measured by determining the amount of force required to displace the target location by 4 mm, wherein the peak load is measured within the 4 mm displacement. In some embodiments, the tracking tip 1210 can have a peak load value between about 20 gF to about 30 gF, between about 22 gF to about 28 gF, between about 24 gF to about 26 gF, and any value in between the ranges listed including endpoints. In some embodiments, the tracking tip 1210 can have a peak load value of 18 gF, between about 16 gF to about 20 gF, between about 14 gF to about 22 gF, between about 12 gF to about 24 gF, between about 10 gF to about 26 gF, and any value in between the ranges listed including endpoints.

Section 2200 illustrates the flexibility profile along the length of the increasing stiffness portion 1220. Section 2200 represents the peak load along the length of the increasing stiffness portion 1220 measured by determining the amount of force required to displace the target location by 4 mm, wherein the peak load is measured within the 4 mm displacement. In some embodiments, the increasing stiffness portion 1220 can change in peak load from a first peak load to a second peak load. The first peak load can range between about 20 gF to about 30 gF, between about 22 gF to about 28 gF, between about 24 gF to about 26 gF, and any values in between the ranges listed including endpoints The second peak load can range between about 50 gF to about 70 gF, between about 52 gF to about 68 gF, between about 54 gF to about 66 gF, between about 56 gF to about 64 gF, between about 60 gF to about 65 gF, and any values in between the ranges listed including endpoints.

Section 2300 illustrates the flexibility profile along the length of the transition portion 1300. Section 2300 represents the peak load along the length of the transitional portion 1300 measured by determining the amount of force required to displace the target location by 4 mm, wherein the peak load is measured within the 4 mm displacement. In some embodiments, the transitional portion 1300 can have a peak load value between about 50 gF to about 70 gF, between about 52 gF to about 68 gF, between about 54 gF to about 66 gF, between about 56 gF to about 64 gF, between about 60 gF to about 70 gF, and any value in between the ranges listed including endpoints.

Section 2400 illustrates the flexibility profile along the length of the support portion 1400. Section 2400 represents the peak load along the length of the support portion 1400 measured by determining the amount of force required to displace the target location by 4 mm, wherein the peak load is measured within the 4 mm displacement. In some embodiments, the support portion 1400 can change in peak load from a first peak load to a second peak load. The first peak load can range between about 50 gF to about 70 gF, between about 52 gF to about 68 gF, between about 54 gF to about 66 gF, between about 56 gF to about 64 gF, between about 58 gF to about 62 gF, between about 60 gF to about 70 gF, and any value in between the ranges listed including endpoints. The second stiffness is at least about 400 gF, between about 500 gF to about 550 gF, between about 510 gF to about 540 gF, between about 520 gF to about 530 gF, and any value in between the ranges listed including endpoints.

FIG. 6B illustrates a schematic of the catheter flexibility profile between the distal tracking tip 1210 and the transitional portion 1300 of the distal segment 1100. In particular, FIG. 6B highlights the importance of the rate of change of the flexibility of the catheter. As the catheter is designed to match the anatomy of the patient, a change of flexibility that is either too fast or too slow can render the catheter ineffective in traversing the patient's intracranial vasculature.

As discussed above with regard to FIG. 6B, section 2100 represents the flexibility profile of the tracking tip 1200. As shown, section 2100 can have no change or substantially no change in flexibility (e.g., a slope of 0) because the distal portion 1200 (in particularly the tracking tip 1210) is designed to be sufficiently soft such that when it hits the wall it buckles and turns. Section 2300, represents the flexibility profile of the transitional portion 1300. In some embodiments, the transitional portion 2300 does not have a change in flexibility as it forms a “soft lock” in the petrous segment of the internal carotid artery.

Section 2200 represents the flexibility profile of the increasing stiffness portion 1220 as the catheter material transitions between the tracking tip 1210 and the transitional portion 1300. The slope of section 2200 is can ensure that the catheter performs properly. In some embodiments, a slope of section 2200 that is too high or too low can result in a poor performing catheter. Section 2200b illustrates the slope for section 2200 wherein the transitional portion 1300 provides sufficient support to the catheter 1000 to navigate to the target anatomy and ensure 1:1 translation of the hand motion to catheter tip advancement. As shown in FIG. 6A, segment 2200 illustrates that the stiffness of the increasing stiffness portion 1220 can gradually increase from a first stiffness to a second stiffness over the length of the increasing stiffness portion 1220. In some embodiments, the stiffness of the increasing stiffness portion 1220 can gradually increase from below about 30 gF to around about 50 gF to about 70 gF over at least a length of about 12 mm. In some embodiments, the increasing stiffness portion 1220 can have a first starting stiffness of between about 0 gF to about 50 gF, between about 10 gF to about 40 gF, between about 15 gF, to about 35 gF, between about 17 gF to about 33 gF and any value in between the ranges listed including endpoints. In some embodiments, the increasing stiffness portion 1220 can have a second ending stiffness of about 60 gF, between about 55 gF to about 65 gF, between about 50 gF to about 70 gF, between about 45 gF to about 75 gF, between about 40 gF to about 80 gF, and any value in between the ranges listed including endpoints.

By contrast, sections 2200a and 2200c reflect flexibility profiles that are either too high or too low for the transitional portion 1300. Section 2200a illustrates a slope that is too high (e.g., the change in flexibility is too high and the stiffness is too great). A slope that is too high can increase the risk that the catheter will kink. Section 2200c illustrates a slope that is too low (e.g., the change in flexibility is too low). A slope that is too low can translate into poor force transmission and may cause the catheter to move to the outside of the curve, prolapse, or snake within the vessel. When the catheter has insufficient support, the mechanical feedback of the catheter will feel spongy to the doctor and may fail to provide 1:1 translation of the hand motion to catheter tip advancement.

### Material Properties of Distal Segment

FIG. 7 illustrates a graph of durometer or modulus along the length of the presently disclosed catheter, from the proximal end (x=0) to the distal end (x=1). The catheter according to an embodiment may have a decreasing durometer or modulus I approaching its distal end. The proximal end of the catheter has a higher durometer or modulus than that of the distal end of the catheter. High durometer or modulus near the proximal end provides superior back-up support of the catheter. The durometer or modulus of the catheter is substantially constant along its length near the proximal segment 1500 of the catheter. Then, durometer or modulus of the catheter decreases near the distal segment 1100. The durometer or modulus of the catheter may begin to decrease (i.e., transition region) at about 50%, 70%, 75%, 80%, or 90% of the length of the catheter from its proximal end. The catheter may have successively decreasing durometer or modulus near its distal end by using materials with less durometer or modulus or having a thinner catheter wall near the distal end. Decreased durometer or modulus near the distal end provides superior trackability of the catheter. In some embodiments, the catheters can have a flexural load that is substantially constant along the longitudinal length near the proximal end and a rapidly decreasing flexural load near the distal end.

In some embodiments, the distal portion 1200 of the distal segment can have a durometer of between about 40A to about 73A, between about 42A to about 70A, between about 44A to about 68A, between about 48A to about 66A, between about 50A to about 64A, or about 62A and any value in between the ranges listed, including endpoints.

In some examples, the transitional portion 1300 can comprise a material such as urethane. In some embodiments, the transitional portion 1300 can more than one material having differing hardness. In some example, the distal end of the transitional portion 1300 can have a durometer of between about 60D to about 84D, between about 62D to about 82D, between about 64D to about 80D, between about 66D to about 78D, between about 68D to about 76D, between 70D to about 74D, or about 72D and any value in between the ranges listed, including endpoints. In some embodiments, the proximal end of the transitional portion 1300 can have a durometer of between about 60D to about 84D, between about 62D to about 82D, between about 64D to about 80D, between about 66D to about 78D, between about 68D to about 76D, between 70D to about 74D, or about 72D and any value in between the ranges listed, including endpoints. In some embodiments, the proximal end of the transitional portion 1300 can have a wall thickness that is thicker than the wall thickness of the distal end of the transitional portion 1300.

In some embodiments, the support portion 1300 can comprise a polyether block amide. The support portion 1300 can increase in hardness along the length of the support portion 1300 as it extends from a distal end (e.g., adjacent to the transitional portion 1300) to a proximal end (e.g., adjacent to the proximal segment 1500). In some embodiments, the support portion 1300 can comprise a plurality of adjacent segments that increase in hardness. Each of the adjacent segments of the support portion 1300 can have a length between about 0 cm to about 1.6 cm, between about 0.2 cm to about 1.4 cm, between about 0.4 cm to about 1.2 cm, between about 0.6 cm to about 1.0 cm, or about 0.8 cm and any value in between the ranges listed including endpoints. In some examples, each of the adjacent segments of the support portion 1300 can have a length between about 0 cm to about 2 cm, between about 0.2 cm to about 1.8 cm, between about 0.4 cm to about 1.6 cm, between about 0.6 cm to about 1.4 cm, between about 0.8 cm to about 1.2 cm, or about 1.0 cm and any value in between the ranges listed between endpoints.

In some embodiments, each of the segments of the support portion 1300 can have a durometer of between 30D to about 40D, between about 31D to about 39D, between about 32D to about 38D, between about 33D to about 37D, between about 34D to about 36D, or about 35D and any value in between the ranges listed including endpoints. In some embodiments, each of the segments of the support portion 1300 can have a durometer of between about 32D to about 42D, between about 33D to about 41D, between about 34D to about 40D, between about 35D to about 39D, between 36D to about 38D, or about 37D and any value in between the ranges listed including endpoints. In some embodiments, each of the segments of the support portion 1300 can have a durometer of between 35D to about 45D, between about 36D to about 44D, between about 37D to about 43D, between about 36D to about 42D, between about 35D to about 41D, or about 40D and any value in between the ranges listed including endpoints. In some embodiments, each of the segments of the support portion 1300 can have a durometer of between about 42D to about 52D, between about 43D to about 51D, between about 44D to about 50D, between about 45D to about 49D, between about 46D to about 48D, or about 47D and any value in between the ranges listed including endpoints. In some embodiments, each of the segments of the support portion 1300 can have a durometer of between 50D to about 60D, between about 51D to about 59D, between about 52D to about 58D, between about 53D to about 57D, between about 54D to about 56D, or about 55D and any value in between the ranges listed including endpoints. In some embodiments, each of the segments of the support portion 1300 can have a durometer of between about 54D to about 64D, between about 55D to about 63D, between about 56D to about 62D, between about 57D to about 61D, between about 58D to about 60D, or about 59D and any value in between the ranges listed including endpoints. In some embodiments, each of the segments of the support portion 1300 can have a durometer of between about 58D and about 68D, between about 59D and about 67D, between about 60D and about 66D, between about 61D and about 65D, between about 62D and about 64D, or about 63D and any value in between the ranges listed including endpoints. In some embodiments, each of the segments of the support portion 1300 can have a durometer of between about 67D to about 77D, between about 68D to about 76D, between about 69D to about 75D, between about 70D to about 74D, between about 71D to about 73D, or about 72D and any value in between the ranges listed including endpoints.

### Treatment of Tandem Lesions

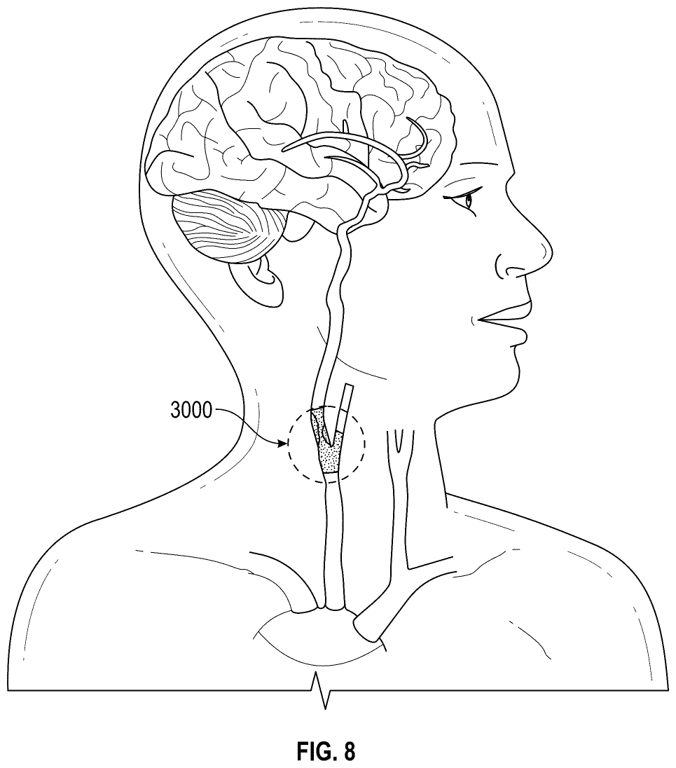

Tandem lesions most commonly occur at the external carotid artery (ECA) bifurcation and can be treated using carotid stenting. FIG. 8 illustrates a graphic illustrating the position of a tandem lesion 3000 in the external carotid artery. The ECA bifurcation to the arch has a segment length that is, on average, about 14 cm long.

The disclosed catheter can also be designed to complement the anatomy of the artery such that it is configured to treat tandem lesions. In some embodiments, the disclosed catheter has enough support to treat the tandem occlusion by delivering a carotid stent when the tip of the catheter is placed at the ECA bifurcation. Because the distance between the ECA bifurcation and the arch for many patients is approximately 14 cm, the distal segment 1100 of the disclosed catheter intended for those patients has an effective length of about 14 cm but no more than about 18 cm. The effective length of the distal segment 1100 allows for the catheter to be locked into the arch during carotid stent delivery. It also lowers the risk of prolapsing in the arch during stent delivery.

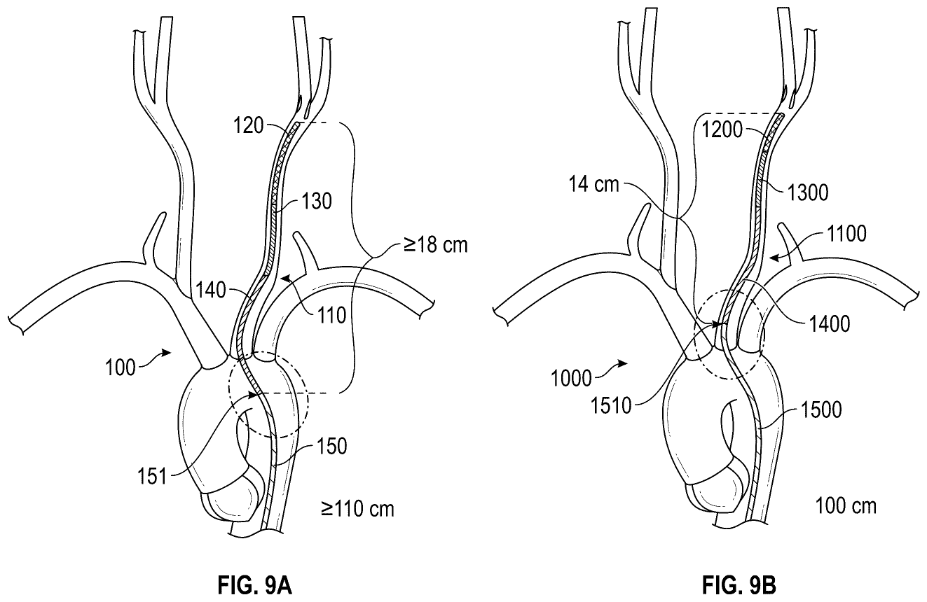

FIGS. 9A-9B illustrate the effectiveness of a catheter 100 having a longer overall length (e.g., at least 110 cm) and a longer distal segment 110 (e.g., at least 18 cm) when compared to the disclosed catheter 1000 (e.g., an overall length of about 100 cm) and a shorter distal segment 1100 (e.g., about 14 cm). In FIG. 9A, the proximal end 151 of the support portion 140 of the catheter 100 is positioned lower than the proximal end 1510 of the support portion 1400 of the catheter 1000 shown in FIG. 9B.

FIG. 9A illustrates the longer catheter 100 positioned between the ECA bifurcation and the arch. As shown, the transitional portion 130 of the catheter 100 is not properly locked into the arch. As a result, there is a risk of prolapsing the distal portion 120 down the ascending aorta and losing or compromising arch access during the stent delivery as the catheter 100 provides insufficient support. FIG. 9B illustrates the catheter 1000 having a shorter effective length (e.g., approximately 100 cm) and a shorter distal flexible segment (e.g., approximately 14 cm). The transition segment 1300 of the catheter 1000 of FIG. 9B is placed such that the proximal end of the support portion 1400 is locked into the arch. This lowers the risk of prolapsing in the arch during stent delivery. This can ensure that the catheter will provide sufficient support when the carotid stent is delivered to the tandem lesion.

### Other Implementations

In some implementations, access for the catheter can be achieved using conventional techniques through an incision on a peripheral artery, such as right femoral artery, left femoral artery, right radial artery, left radial artery, right brachial artery, left brachial artery, right axillary artery, left axillary artery, right subclavian artery, or left subclavian artery. An incision can also be made on the right carotid artery or left carotid artery in emergency situations.

Avoiding a tight fit between a guidewire and inside diameter of guidewire lumen enhances the slidability of the catheter over the guidewire. In ultra small diameter catheter designs, it may be desirable to coat the outside surface of the guidewire and/or the inside surface of the wall defining lumen with a lubricous coating to minimize friction as the catheter 1000 is axially moved with respect to the guidewire. A variety of coatings may be utilized, such as Paralene, Teflon, silicone, polyimide-polytetrafluoroethylene composite materials or others known in the art and suitable depending upon the material of the guidewire or inner tubular wall.

In some examples, the catheters may be composed of any of a variety of biologically compatible polymeric resins having suitable characteristics when formed into the tubular catheter body segments. Exemplary materials include polyvinyl chloride, polyethers, polyamides, polyethylenes, polyurethanes, copolymers thereof, and the like.

The proximal body segment will exhibit sufficient column strength to permit axial positioning of the catheter through a guide catheter with at least a portion of the proximal body segment extending beyond the guide catheter and into the patient's vasculature.

The catheter body may further comprise other components, such as radiopaque fillers; colorants; reinforcing materials; reinforcement layers, such as braids or helical reinforcement elements; or the like. In particular, the proximal body segment may be reinforced in order to enhance its column strength and torqueability (torque transmission) while preferably limiting its wall thickness and outside diameter.

In accordance with one aspect, there is provided a method of aspirating a vascular occlusion from a remote site, comprising: advancing a guidewire to a site at least as distal as the cavernous segment of the internal carotid artery; advancing a tubular body directly over the guidewire to a site at least as distal as the cavernous segment; removing the guidewire from the tubular body; and aspirating thrombus into the tubular body by applying vacuum to the tubular body. In one aspect of present disclosure, the method of aspirating a vascular occlusion comprises advancing the tubular body at least as distal as the cerebral segment of the internal carotid artery. In another aspect of present disclosure, the method of aspirating a vascular occlusion comprises advancing the guidewire at least as distal as the middle cerebral artery.

In yet another aspect of present disclosure, the method of aspirating a vascular occlusion further comprises providing sufficient back up support to the tubular body to resist prolapse of the tubular body into the aorta. In one aspect, the back up support may be provided by advancing the tubular body over a guidewire having a distal end positioned at least as distal as the cavernous segment of the internal carotid artery, and a diameter at the point the guidewire enters the brachiocephalic artery of at least about 0.030 inches. In another aspect, the back up support may be provided by advancing the tubular body over a guidewire having a distal end positioned at least as distal as the cavernous segment of the internal carotid artery, and a diameter at the point the guidewire enters the brachiocephalic artery of about 0.038 inches. The guidewire may be navigable to at least the cerebral segment of the internal carotid artery by having a distal segment having a diameter of no more than about 0.020 inches. The guidewire may be navigable to at least the cerebral segment of the internal carotid artery by having a distal segment having a diameter of about 0.016 inches. The distal segment may have a length of no more than about 25 cm. The distal segment may have a length of no more than about 20 cm.

In accordance with one aspect, there is provided a method of aspirating a vascular occlusion from a remote site, comprising: advancing a guidewire through a vascular access point and transvascularly to a site at least as distal as the cavernous segment of the internal carotid artery; accessing a site at least as distal as the cavernous segment by advancing a combined access and aspiration catheter directly over the guidewire; removing the guidewire; and aspirating thrombus through the combined access and aspiration catheter. In one aspect of present disclosure, the method of aspirating a vascular occlusion comprises advancing the combined access and aspiration catheter at least as distal as the cerebral segment of the internal carotid artery. In another aspect of present disclosure, the method of aspirating a vascular occlusion comprises advancing the guidewire at least as distal as the middle cerebral artery.

### Disclaimer

Although the present invention has been described in terms of certain preferred embodiments, it may be incorporated into other embodiments by persons of skill in the art in view of the disclosure herein. The scope of the invention is therefore not intended to be limited by the specific embodiments disclosed herein but is intended to be defined by the full scope of the following claims.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The drawings are for the purpose of illustrating embodiments of the invention only, and not for the purpose of limiting it.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they Can also include any third-party instruction of those actions, either expressly or by implication. For example, actions such as “deploying an instrument sterilized using the systems herein” include “instructing the deployment of an instrument sterilized using the systems herein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

Any titles or subheadings used herein are for organization purposes and should not be used to limit the scope of embodiments disclosed herein.

The terms “approximately”, “about”, and “substantially” as used herein represent an amount or characteristic close to the stated amount or characteristic that still performs a desired function or achieves a desired result. For example, the terms “approximately”, “about”, and “substantially” may refer to an amount in certain embodiments that is within less than plus or minus 10% of, within less than plus or minus 5% of, within less than plus or minus 1% of, within less than plus or minus 0.1% of, and within less than plus or minus 0.01% of the stated amount or characteristic.

### Example Embodiments

Embodiment 1: A neurovascular catheter for insertion into an internal carotid artery of a patient, the neurovascular catheter comprising:
an elongate flexible body comprising a length of less than about 110 cm, the elongate flexible body comprising:
   a flexible distal portion comprising a length of between about 10 cm and about 18 cm, the flexible distal portion comprising:
      a distal tip configured to be positioned within a cavernous segment of the internal carotid artery;
      a transitional portion proximal to the distal tip that is less flexible than the distal tip, wherein the transitional portion is configured to be positioned within a petrous segment of the internal carotid artery; and
      a support portion proximal to the transitional portion that is less flexible than the transitional portion, wherein the support portion is configured to extend proximal to a base of the patient's skull.

Embodiment 2: The catheter of Embodiment 1, wherein the elongate flexible body has a length of between about 98 cm and about 102 cm.

Embodiment 3: The catheter of any of Embodiments 1-2, wherein the flexible distal portion has a length of between about 12 cm and about 16 cm.

Embodiment 4: The catheter of any of Embodiments 1-3, wherein the distal tip comprises a hardness of between about 40A to about 73A.

Embodiment 5: The catheter of any of Embodiments 1-4, wherein the distal tip has a length of between about 15 mm to about 20 mm.

Embodiment 6: The catheter of any of Embodiments 1-5, wherein the distal tip has a length of between about 10 mm to about 20 mm.

Embodiment 7: The catheter of any of Embodiments 1-6, wherein the transitional portion comprises urethane.

Embodiment 8: The catheter of any of Embodiments 1-7, wherein the transitional portion has a length of between about 1.0 cm and 3.5 cm.

Embodiment 9: The catheter of any of Embodiments 1-8, wherein the proximal end of the transitional portion is positioned a distance of between about 5.5 cm and about 7.5 cm from a distal end of the flexible distal portion.

Embodiment 10: The catheter of any of Embodiments 1-9, wherein at least a portion of the support portion comprises a durometer ranging between 35D and 72D.

Embodiment 11: The catheter of any of Embodiments 1-10, wherein the support portion comprises a polymer block amide.

Embodiment 12: The catheter of any of Embodiments 1-11, wherein the distal tip comprises a constant stiffness along a length of the distal tip.

Embodiment 13: The catheter of Embodiment 12, wherein the stiffness is between about 20 gF to about 30 gF.

Embodiment 14: The catheter of any of Embodiments 1-13, wherein the flexible distal portion further comprises a flexible portion that extends proximal to the distal tip and comprises a stiffness that increases along a length of the flexible portion.

Embodiment 15: The catheter of Embodiment 12, wherein the flexible portion has a first stiffness at a first end of the flexible portion that is between about 20 gF to about 30 gF, and wherein the flexible portion has a second stiffness at a second end of the flexible portion that is between about 60 gF to about 65 gF.

Embodiment 16: The catheter of any of Embodiments 1-15, wherein the transitional portion comprises a constant stiffness along a length of the distal tip.

Embodiment 17: The catheter of Embodiment 16, wherein the stiffness is between about 50 gF to about 70 gF.

Embodiment 18: The catheter of any of Embodiments 1-17, wherein the support portion comprises a stiffness that increases along a length of the support portion.

Embodiment 19: The catheter of Embodiment 18, wherein the support portion has a first stiffness at a first end of the support portion that is between about 60 gF to about 70 gF, and wherein the support portion has a second stiffness at a second end of the support portion that is at least 400 gF.

Embodiment 20: A method for accessing the petrous-cavernous segment of a patient, wherein the petrous-cavernous segment comprises a length between 2.9 cm and 5.4 cm, the method comprising:
providing a catheter comprising an elongate flexible body, a flexible distal portion comprising a distal tip, a transitional portion, and a support portion; and
advancing the distal tip of the catheter into an internal carotid artery of a patient until the distal tip of the flexible distal portion is positioned within a cavernous segment of the internal carotid artery, the transitional portion is positioned within a petrous segment of the internal carotid artery, wherein the transitional portion is proximal to the distal tip and is less flexible than the distal tip, and the support portion extends from a base of the patient's skull, wherein the support portion is proximal to the transitional portion and is less flexible than the transitional portion.

Embodiment 21: The method of Embodiment 20, wherein the distal tip of the guide catheter is inserted until a tactile feedback is received at a proximal end of the elongate flexible body indicating that the transitional portion has engaged with the anatomy of the petrous segment.

Embodiment 22: The method of any of Embodiments 20-21, wherein the elongate flexible body comprises a length of less than 110 cm.

Embodiment 23: The method of any of Embodiments 20-22, wherein the elongate flexible body comprises a length of 100 cm.

Embodiment 24: The method of Embodiment 20, wherein the flexible distal portion comprises a length of between 14 cm and 18 cm.

Embodiment 25: The method of Embodiment 20, wherein the flexible distal portion comprises a length of 14 cm.

Embodiment 26: The method of Embodiment 20, wherein the transitional portion comprises a length of between about 1.0 cm and 3.5 cm.

Embodiment 27: The method of Embodiment 20, wherein the proximal end of the transitional portion is positioned a distance of 6.5 cm from a distal end of the flexible distal portion.

Embodiment 28: The method of Embodiment 20, wherein the distal tip comprises a length of 17 mm.

Embodiment 29: The method of Embodiment 20, wherein the distal tip is softer than the transitional portion, and wherein the transitional portion is softer than the support portion.

Embodiment 30: The method of Embodiment 20, wherein the method is configured to treat a tandem lesion.

Embodiment 31: A neurovascular catheter for insertion into an internal carotid artery of a patient, the neurovascular catheter comprising:
an elongate flexible body comprising a length of less than about 110 cm, the elongate flexible body comprising:
   a flexible distal segment comprising a length of between about 10 cm and about 18 cm, the flexible distal segment comprising:
      a distal portion having a length between about 20 mm and 40 mm, the distal portion comprising:
         a tracking tip configured to be positioned within a cavernous segment of the internal carotid artery, wherein the tracking tip has a length between about 10 mm and about 20 mm; and
         an increasing stiffness portion proximal to the tracking tip, wherein the increasing stiffness portion has a length between about 5 mm and about 15 mm;
      a transitional portion proximal to the distal portion that is less flexible than the tracking tip, wherein the transitional portion is configured to be positioned within a petrous segment of the internal carotid artery, wherein the transitional portion has a length of between about 30 mm and about 40 mm; and
      a support portion proximal to the transitional portion that is less flexible than the transitional portion, wherein the support portion is configured to extend proximal to a base of a patient's skull, wherein the support portion comprises a stiffness that increases proximally along a length of the support portion, wherein the support portion has a length between about 70 mm and about 80 mm;
   wherein a flexibility profile of the flexible distal segment is measurable with a cantilever beam test with a 5 mm gage length and 4 mm displacement to determine a peak load value, and wherein:
      the peak load value in the tracking tip is between about 10 gF and about 30 gF;
      the peak load value in the increasing stiffness portion increases from between about 20 gF and about 30 gF to between about 50 gF and about 70 gF over the length of the increasing stiffness portion;
      the peak load value in the transitional portion is between about 50 gF and about 70 gF; and
      the peak load value at a first end of the support portion is between about 50 gF and about 70 gF and at a second end of the support portion is at least about 400 gF.

Embodiment 32: The neurovascular catheter of Embodiment 31, wherein the elongate flexible body has a length of about 100 cm.

Embodiment 33: The neurovascular catheter of any of Embodiments 31-32, wherein the flexible distal segment has a length of about 14 cm.

Embodiment 34: The neurovascular catheter of any of Embodiments 31-33, wherein the distal portion has a length of about 30 mm.

Embodiment 35: The neurovascular catheter of any of Embodiments 31-34, wherein the tracking tip has a length of about 17 mm.

Embodiment 36: The neurovascular catheter of any of Embodiments 31-35, wherein the increasing stiffness portion has a length of about 13 mm.

Embodiment 37: The neurovascular catheter of any of Embodiments 31-36, wherein the transitional portion has a length of about 35 mm.

Embodiment 38: The neurovascular catheter of any of Embodiments 31-37, wherein the support portion has a length of about 75 mm.

Embodiment 39: A neurovascular catheter for insertion into an internal carotid artery of a patient, the neurovascular catheter comprising:
a flexible distal segment of the neurovascular catheter for insertion into an internal carotid artery of a patient, the flexible distal segment comprising:
   a distal portion comprising:
      a tracking tip configured to be positioned within a cavernous segment of the internal carotid artery, and
      an increasing stiffness portion;
   a transitional portion configured to be positioned within a petrous segment of the internal carotid artery;
   a support portion configured to extend proximally to a base of a patient's skull;
wherein a flexibility profile of the flexible distal segment is measurable with a cantilever beam test with a 5 mm gage length and 4 mm displacement to determine a peak load value, and wherein:
   the peak load value in the tracking tip is less than about 30 gF;
   the peak load value in the increasing stiffness portion increases from between about 20 gF and about 30 gF to between about 50 gF and about 70 gF over a length of the increasing stiffness portion;
   the peak load value in the transitional portion is between about 50 gF and about 70 gF; and
   the peak load value at a first end of the support portion is between about 50 gF and about 70 gF and at a second end of the support portion is at least about 400 gF.

Embodiment 40: The neurovascular catheter of Embodiment 39, wherein the flexible distal segment has a length of between about 10 cm and about 18 cm.

Embodiment 41: The neurovascular catheter of Claim 40, wherein the flexible distal segment has a length of about 14 cm.

Embodiment 42: The neurovascular catheter of any of Embodiments 39-41, wherein the distal portion has a length between about 20 mm and 40 mm.

Embodiment 43: The neurovascular catheter of any of Embodiments 39-42, wherein the distal portion has a length of about 30 mm.

Embodiment 44: The neurovascular catheter of any of Embodiments 39-43, wherein the tracking tip has a length between about 10 mm and about 20 mm.

Embodiment 45: The neurovascular catheter of Embodiment 44, wherein the tracking tip has a length of about 17 mm.

Embodiment 46: The neurovascular catheter of any of Embodiments 39-45, wherein the increasing stiffness portion has a length between about 5 mm and about 15 mm.

Embodiment 47: The neurovascular catheter of Embodiment 46, wherein the increasing stiffness portion has a length of about 13 mm.

Embodiment 48: The neurovascular catheter of any of Embodiments 39-47, wherein the transitional portion has a length of between about 30 mm and about 40 mm.

Embodiment 49: The neurovascular catheter of Embodiment 48, wherein the transitional portion has a length of about 35 mm.

Embodiment 50: The neurovascular catheter of any of Embodiments 39-49, wherein the support portion has a length between about 70 mm and about 80 mm.

Embodiment 51: The neurovascular catheter of Embodiment 50, wherein the support portion has a length of about 75 mm.

Embodiment 52: A method for accessing a petrous-cavernous segment of a patient, the method comprising:
providing a catheter comprising a flexible distal segment comprising a distal portion having a tracking tip and an increasing stiffness portion, a transitional portion, and a support portion;
advancing the flexible distal segment of the catheter into an internal carotid artery of a patient such that:
   the tracking tip of the flexible distal segment is positioned within a cavernous segment of the internal carotid artery,
   the transitional portion is positioned within a petrous segment of the internal carotid artery, and
   the support portion extends to a base of a patient's skull.

Embodiment 53: The method of Embodiment 52, wherein the tracking tip of the flexible distal segment of the catheter is inserted until a tactile feedback is received at a proximal end of the catheter indicating that the transitional portion has engaged with an anatomy of the petrous segment.

Embodiment 54: The method of any of Embodiments 52-53, wherein the catheter has a length of less than 110 cm.

Embodiment 55: The method of any of Embodiments 52-54, wherein the catheter has a length of 100 cm.

Embodiment 56: The method of any of Embodiments 52-55, wherein the flexible distal segment has a length of between 10 cm and 18 cm.

Embodiment 57: The method of any of Embodiments 52-56, wherein the flexible distal segment has a length of 14 cm.

Embodiment 58: The method of any of Embodiments 52-57, wherein the distal portion has a length between about 20 mm and 40 mm.

Embodiment 59: The method of any of Embodiments 52-58, wherein the distal portion has a length of about 30 mm.

Embodiment 60: The method of any of Embodiments 52-59, wherein the tracking tip has a length between about 10 mm and about 20 mm.

Embodiment 61: The method of any of Embodiments 52-60, wherein the tracking tip has a length of about 17 mm.

Embodiment 62: The method of any of Embodiments 52-61, wherein the increasing stiffness portion has a length between about 5 mm and about 15 mm.

Embodiment 63: The method of any of Embodiments 52-62, wherein the increasing stiffness portion has a length of about 13 mm.

Embodiment 64: The method of any of Embodiments 52-63, wherein the transitional portion has a length of between about 30 mm and about 40 mm.

Embodiment 65: The method of any of Embodiments 52-64, wherein the transitional portion has a length of about 35 mm.

Embodiment 66: The method of any of Embodiments 52-65, wherein the support portion has a length between about 70 mm and about 80 mm.

Embodiment 67: The method of any of Embodiments 52-66, wherein the support portion has a length of about 75 mm.

Embodiment 68: The method of any of Embodiments 52-67, wherein the distal tip is softer than the transitional portion, and wherein the transitional portion is softer than the support portion.

Embodiment 69: The method of any of Embodiments 52-68, wherein the method is configured to treat a tandem lesion.

Embodiment 70: The method of any of Embodiments 52-69, wherein a flexibility profile of the flexible distal segment is measurable with a cantilever beam test with a 5 mm gage length and 4 mm displacement to determine a peak load value, and wherein:
the peak load value in the tracking tip is between about 10 gF and about 30 gF;
the peak load value in the increasing stiffness portion increases from between about 20 gF and about 30 gF to between about 50 gF and about 70 gF over a length of the increasing stiffness portion;
the peak load value in the transitional portion is between about 50 gF and about 70 gF; and
the peak load value at a first end of the support portion is between about 50 gF and about 70 gF and at a second end of the support portion is at least about 400 gF.

1. A neurovascular catheter for insertion into an internal carotid artery of a patient, the neurovascular catheter comprising:
an elongate flexible body comprising a length of less than about 110 cm, the elongate flexible body comprising:
a flexible distal portion comprising a length of between about 10 cm and about 18 cm, the flexible distal portion comprising:
a distal tip configured to be positioned within a cavernous segment of the internal carotid artery;
a transitional portion proximal to the distal tip that is less flexible than the distal tip, wherein the transitional portion is configured to be positioned within a petrous segment of the internal carotid artery; and
a support portion proximal to the transitional portion that is less flexible than the transitional portion, wherein the support portion is configured to extend proximal to a base of the patient's skull.

2. The catheter of claim 1, wherein the elongate flexible body has a length of between about 98 cm and about 102 cm.

3. The catheter of claim 1, wherein the flexible distal portion has a length of between about 12 cm and about 16 cm.

4. The catheter of claim 1, wherein the distal tip has a length of between about 15 mm to about 20 mm.

5. The catheter of claim 1, wherein the transitional portion has a length of between about 1.0 cm and 3.5 cm.

6. The catheter of claim 1, wherein a proximal end of the transitional portion is positioned a distance of between about 5.5 cm and about 7.5 cm from a distal end of the flexible distal portion.

7. The catheter of claim 1, wherein the distal tip comprises a substantially constant stiffness along a length of the distal tip, wherein the substantially constant stiffness is between about 20 gF to about 30 gF, wherein the substantially constant stiffness is determined using a cantilever beam test with a 5 mm gage length and 4 mm displacement to determine a peak load value.

8. (canceled)

9. The catheter of claim 1, wherein the flexible distal portion further comprises a flexible portion that extends proximal to the distal tip and comprises a stiffness that increases along a length of the flexible portion, wherein the flexible portion has a first stiffness at a first end of the flexible portion that is between about 20 gF to about 30 gF, and wherein the flexible portion has a second stiffness at a second end of the flexible portion that is between about 60 gF to about 65 gF.

10. (canceled)

11. The catheter of claim 1, wherein the transitional portion comprises a substantially constant stiffness along a length of the distal tip, and wherein the substantially constant stiffness is between about 50 gF to about 70 gF.

12. (canceled)

13. The catheter of claim 1, wherein the support portion comprises a stiffness that increases along a length of the support portion, wherein the support portion has a first stiffness at a first end of the support portion that is between about 60 gF to about 70 gF, and wherein the support portion has a second stiffness at a second end of the support portion that is at least 400 gF.

14. (canceled)

15. A neurovascular catheter for insertion into an internal carotid artery of a patient, the neurovascular catheter comprising:
an elongate flexible body comprising a length of less than about 110 cm, the elongate flexible body comprising:
a flexible distal segment comprising a length of between about 10 cm and about 18 cm, the flexible distal segment comprising:
a distal portion having a length between about 20 mm and 40 mm, the distal portion comprising:
a tracking tip configured to be positioned within a cavernous segment of the internal carotid artery, wherein the tracking tip has a length between about 10 mm and about 20 mm; and
an increasing stiffness portion proximal to the tracking tip, wherein the increasing stiffness portion has a length between about 5 mm and about 15 mm;
a transitional portion proximal to the distal portion that is less flexible than the tracking tip, wherein the transitional portion is configured to be positioned within a petrous segment of the internal carotid artery, wherein the transitional portion has a length of between about 30 mm and about 40 mm; and
a support portion proximal to the transitional portion that is less flexible than the transitional portion, wherein the support portion is configured to extend proximal to a base of a patient's skull, wherein the support portion comprises a stiffness that increases proximally along a length of the support portion, wherein the support portion has a length between about 70 mm and about 80 mm;
wherein a flexibility profile of the flexible distal segment is measurable with a cantilever beam test with a 5 mm gage length and 4 mm displacement to determine a peak load value, and wherein:
the peak load value in the tracking tip is less than about 30 gF;
the peak load value in the increasing stiffness portion increases from between about 20 gF and about 30 gF to between about 50 gF and about 70 gF over the length of the increasing stiffness portion;
the peak load value in the transitional portion is between about 50 gF and about 70 gF; and
the peak load value at a first end of the support portion is between about 50 gF and about 70 gF and at a second end of the support portion is at least about 400 gF.

16. (canceled)

17. The neurovascular catheter of claim 15, wherein the flexible distal segment has a length of about 14 cm.

18. The neurovascular catheter of claim 15, wherein the distal portion has a length of about 30 mm.

19. The neurovascular catheter of claim 15, wherein the tracking tip has a length of about 17 mm.

20. The neurovascular catheter of claim 15, wherein the increasing stiffness portion has a length of about 13 mm.

21. The neurovascular catheter of claim 15, wherein the transitional portion has a length of about 35 mm.

22. The neurovascular catheter of claim 15, wherein the support portion has a length of about 75 mm.

23. A neurovascular catheter for insertion into an internal carotid artery of a patient, the neurovascular catheter comprising:
a flexible distal segment of the neurovascular catheter for insertion into an internal carotid artery of a patient, the flexible distal segment comprising:
a distal portion comprising:
a tracking tip configured to be positioned within a cavernous segment of the internal carotid artery, and
an increasing stiffness portion;
a transitional portion configured to be positioned within a petrous segment of the internal carotid artery; and
a support portion configured to extend proximally to a base of a patient's skull;
wherein a flexibility profile of the flexible distal segment is measurable with a cantilever beam test with a 5 mm gage length and 4 mm displacement to determine a peak load value, and wherein:
the peak load value in the tracking tip is less than about 30 gF;
the peak load value in the increasing stiffness portion increases from between about 20 gF and about 30 gF to between about 50 gF and about 70 gF over a length of the increasing stiffness portion;
the peak load value in the transitional portion is between about 50 gF and about 70 gF; and
the peak load value at a first end of the support portion is between about 50 gF and about 70 gF and at a second end of the support portion is at least about 400 gF.

24. The neurovascular catheter of claim 23, wherein the flexible distal segment has a length of between about 10 cm and about 18 cm.

25. The neurovascular catheter of claim 23, wherein the distal portion has a length between about 20 mm and 40 mm.

26. The neurovascular catheter of claim 23, wherein the tracking tip has a length between about 10 mm and about 20 mm.

27. The neurovascular catheter of claim 23, wherein the increasing stiffness portion has a length between about 5 mm and about 15 mm.

28. The neurovascular catheter of claim 23, wherein the transitional portion has a length of between about 30 mm and about 40 mm.

29. The neurovascular catheter of claim 23, wherein the support portion has a length between about 70 mm and about 80 mm.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)
